(19) United States
(12) Patent Application Publication
(10) Pub. No.: US 2024/0325028 A1
(43) Pub. Date: Oct. 3, 2024

(54) CLIP SYSTEM

(71) Applicant: OLYMPUS MEDICAL SYSTEMS CORP., Tokyo (JP)

(72) Inventors: Tsutomu Okada, Tachikawa-shi (JP); Tatsunori TSUNETO, Hino-shi (JP)

(21) Appl. No.: 18/596,911

(22) Filed: Mar. 6, 2024

Related U.S. Application Data
(60) Provisional application No. 63/493,100, filed on Mar. 30, 2023.

(51) Int. Cl. A61B 17/122; A61B 17/00; A61B 17/128

(52) U.S. Cl. CPC A61B 17/1222; A61B 17/1285; A61B 2017/00473

(57) ABSTRACT

A clip system may include a cartridge system to accommodate or store a clip unit and an applicator into which the clip unit is configured to be loaded. The clip system may further include a pack in which the cartridge system and the applicator are packaged together.

### CLAIM OF PRIORITY

The present application claims priority based on U.S. Patent Provisional Application No. 63/493,100 filed in the United States on Mar. 30, 2023, the contents of which are incorporated herein by reference.

### TECHNICAL FIELD

The present disclosure relates to a clip system.

### BACKGROUND

In endoscopic treatment, a clip unit capable of ligating a resected site after treatment to stop bleeding may be used. The clip unit may include a clip which holds a resected site and the like and a pressing tube which accommodates the clip and locks the clip in the closed state. The clip unit can be introduced to a treatment position by an applicator or other introducing device that can be inserted through a channel of an endoscope.

United States Patent Application, Publication No. 2007/0112359 (Patent Document 1) describes a cartridge system in which a clip unit can be reloaded into an applicator. The user can reload the clip unit using a cartridge system.

Thus, the cartridge system storing the reloadable clip unit and the applicator are packaged in separate packs. A user may be required to open the applicator and the cartridge system, connect the applicator and the cartridge system, and use the cartridge system to load the clip unit into the applicator.

### SUMMARY

The present inventors have recognized a need, among other things, a need for a system that reduces the work of loading the clip unit into the applicator using the cartridge system after opening the pack and allows the clip unit loaded into the applicator to be used as quickly as possible. The present disclosure is directed to a clip system that requires less work to load a clip unit into an applicator using a cartridge system after opening a pack.

A clip system according to a first aspect of the present disclosure may include a cartridge system to accommodate a clip unit, an applicator into which the clip unit may be loaded, and a pack in which the cartridge system and the applicator are packaged together.

The clip system of the present disclosure may require less work to load the clip unit into the applicator using the cartridge system after opening the pack, and the clip unit loaded into the applicator can be used as quickly as possible.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagram showing a clip system according to a first embodiment.

FIG. 2 is a diagram showing another aspect of the same clip system.

FIG. 3 is a perspective view of a clip introducing device of the same clip system.

FIG. 4 is a perspective view of a clip unit of a cartridge system in the same clip system.

FIG. 5 is a perspective view of the same clip unit with a pressing tube transparently displayed.

FIG. 6 is a perspective view of a cartridge of the same clip unit.

FIG. 7 is a perspective view of the same cartridge.

FIG. 8 is a cross-sectional view of the same cartridge.

FIG. 9 is a diagram showing a compressing portion of the same cartridge.

FIG. 10 is a diagram showing the same compressing portion.

FIG. 11 is a diagram showing a sheath connection portion of the same cartridge.

FIG. 12 is a diagram showing the same sheath connection portion.

FIG. 13 is a perspective view of a regulation member of the same cartridge.

FIG. 14 is a perspective view of the same regulation member gripped by a pair of arms.

FIG. 15 is a plan view of the same regulation member.

FIG. 16 is a plan view of the same regulation member gripped in a first region.

FIG. 17 is a diagram showing an opened sterile pack.

FIG. 18 is a diagram showing a clip introducing device held by a user.

FIG. 19 is a diagram illustrating a method of loading the same clip unit into the same clip introducing device using the same cartridge.

FIG. 20 is a diagram illustrating a method of loading the same clip unit into the same clip introducing device using the same cartridge.

FIG. 21 is a diagram illustrating a method of loading the same clip unit into the same clip introducing device using the same cartridge.

FIG. 22 is a diagram illustrating a method of loading the same clip unit into the same clip introducing device using the same cartridge.

FIG. 23 is a diagram illustrating a method of loading the same clip unit into the same clip introducing device using the same cartridge.

FIG. 24 is a diagram illustrating a method of loading the same clip unit into the same clip introducing device using the same cartridge.

FIG. 25 is a diagram illustrating a method of loading the same clip unit into the same clip introducing device using the same cartridge.

FIG. 26 is a diagram showing the same cartridge from which a sheath of the same clip introducing device is pulled out.

FIG. 27 is a diagram showing the same cartridge from which the sheath of the same clip introducing device is pulled out.

FIG. 28 is a diagram showing the same clip unit introduced into the body.

FIG. 29 is a diagram showing the same clip unit with a pair of arms closed.

FIG. 30 is a diagram showing the same clip unit with a clip locked.

FIG. 31 is a diagram showing the same clip unit from which the same clip is separated.

FIG. 32 is a diagram showing the same clip unit after being broken.

FIG. 33 is a diagram showing a modified example of the same compressing portion.

FIG. 34 is a diagram showing another modified example of the same compressing portion.

FIG. 35 is a diagram showing a cartridge system according to a second embodiment.

FIG. 36 is a diagram showing a casing accommodated in an outer casing of the same cartridge system.

FIG. 37 is a view of a cartridge shown in FIG. 36 when viewed from a proximal end side.

FIG. 38 is a diagram showing the same casing separated from the same outer casing.

FIG. 39 is a view of the same cartridge shown in FIG. 38 when viewed from a proximal end side.

FIG. 40 is a diagram showing the same casing from which a sheath is pulled out.

FIG. 41 is a diagram illustrating a procedure of pulling out the same sheath.

FIG. 42 is a diagram illustrating a procedure of pulling out the same sheath.

FIG. 43 is a diagram illustrating a procedure of pulling out the same sheath.

FIG. 44 is a diagram showing a cartridge system according to a third embodiment.

FIG. 45 is a diagram showing a cartridge when viewed from one side in the width direction.

FIG. 46 is a diagram showing the same cartridge when viewed from the other side in the same width direction.

FIG. 47 is a diagram showing the same cartridge with a sheath inserted into a sheath insertion path.

FIG. 48 is a diagram showing a casing from which the same sheath is pulled out.

FIG. 49 is a diagram showing the same casing from which the same sheath is pulled out.

FIG. 50 is a diagram showing the same casing from which the same sheath is pulled out.

FIG. 51 is a diagram showing a modified example of a casing connection portion.

FIG. 52 is a diagram showing the same casing connection portion when viewed from an arrow in FIG. 51.

FIG. 53 is a diagram showing the same casing connection portion when viewed from an arrow of FIG. 51.

FIG. 54 is a diagram showing a modified example of the same sheath insertion path.

FIG. 55 is a diagram showing another modified example of the same sheath insertion path.

FIG. 56 is a diagram showing another modified example of the same sheath insertion path.

FIG. 57 is a diagram showing a modified example of the same casing connection portion.

FIG. 58 is a diagram showing the same modified example of the same casing connection portion.

FIG. 59 is a diagram showing the same modified example of the same casing connection portion.

FIG. 60 is a diagram showing another modified example of the same casing connection portion.

FIG. 61 is a diagram showing a modified example of the same sheath.

FIG. 62 is a diagram showing the same modified example of the same casing connection portion into which the same modified example of the same sheath is inserted.

FIG. 63 is a diagram showing a modified example of the same compressing portion.

FIG. 64 is a diagram showing the same modified example of the same compressing portion.

FIG. 65 is a diagram showing the same modified example of the same compressing portion.

FIG. 66 is a diagram showing the same modified example of the same compressing portion.

FIG. 67 is a diagram showing the same modified example of the same compressing portion.

FIG. 68 is a diagram showing a cartridge system according to a fourth embodiment.

FIG. 69 is a diagram showing a cartridge in which a locking mechanism is attached to a compressing portion.

FIG. 70 is a diagram illustrating a method of fixing a sheath so that the sheath does not come off from the same cartridge.

FIG. 71 is a diagram illustrating the same method.

FIG. 72 is a diagram illustrating the same method.

FIG. 73 is a diagram illustrating the same method.

FIG. 74 is a diagram showing a modified example of the same compressing portion.

FIG. 75 is a diagram illustrating a method of separating the same locking mechanism from the same casing.

FIG. 76 is a diagram illustrating the same method.

FIG. 77 is a diagram illustrating a method of removing the same cartridge from the same sheath.

FIG. 78 is a diagram illustrating the same method.

FIG. 79 is a diagram showing a modified example of the same locking mechanism.

FIG. 80 is a diagram showing the same modified example of the same locking mechanism.

FIG. 81 is a diagram showing another modified example of the same locking mechanism.

FIG. 82 is a diagram showing the same modified example of the same locking mechanism.

### DETAILED DESCRIPTION

### First Embodiment

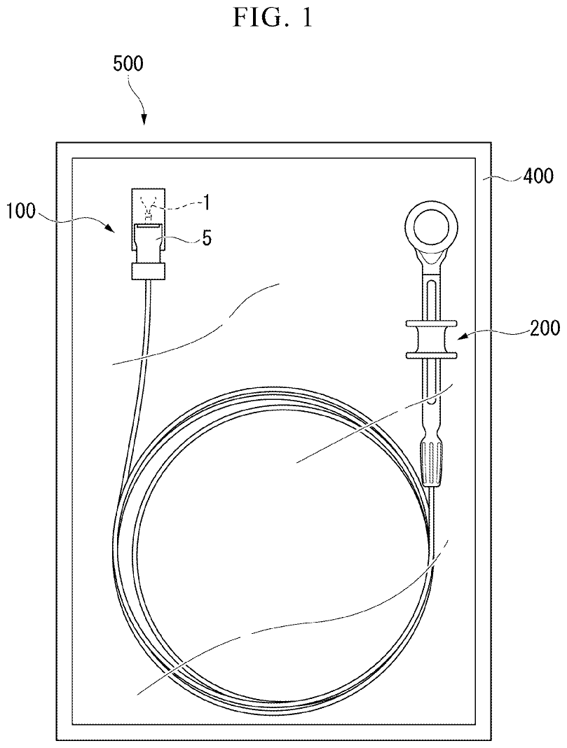

A first embodiment of the present disclosure will be described with reference to FIGS. 1 to 32.

FIG. 1 is a diagram showing a clip system 500 according to this embodiment.

The clip system 500 may include a cartridge system (cartridge) 100, a clip introducing device (e.g., an applicator) 200, and a sterile pack 400. Both the cartridge system 100 and the clip introducing device 200 can be packaged in the sterile pack 400. The cartridge system 100 can be packaged in the sterile pack 400 while being connected to the clip introducing device 200. Specifically, the cartridge system 100 can be connected to the clip introducing device 200 while a clip unit (clip) 1 can be loaded into the clip introducing device 200.

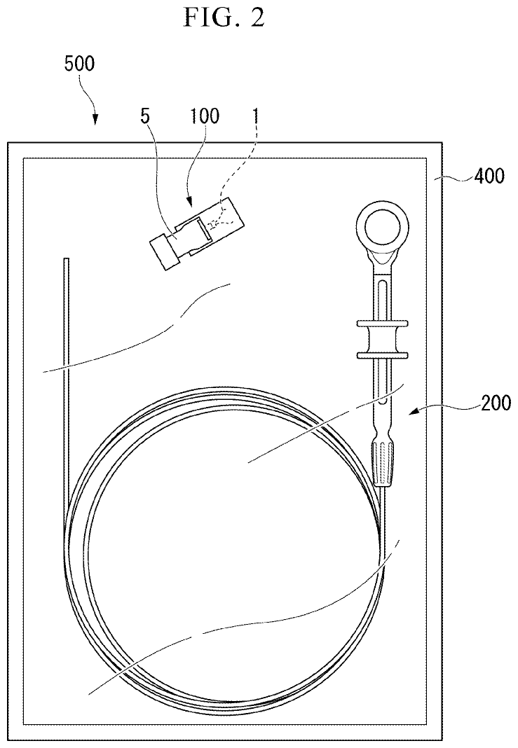

FIG. 2 is a diagram showing another aspect of the clip system 500.

The cartridge system 100 can be packaged in the sterile pack 400 while not being connected to the clip introducing device 200.

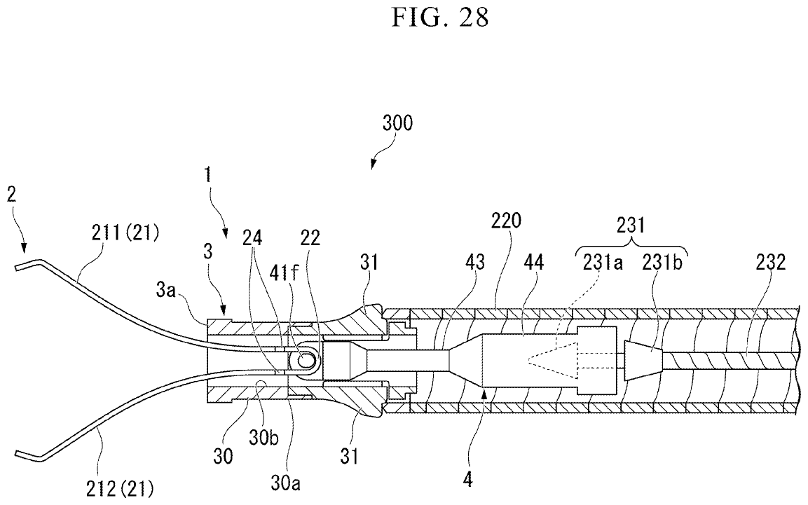

The cartridge system 100 may include the clip unit 1 and a cartridge 5 which accommodates (stores) the clip unit 1. The cartridge system 100 may be a support system for easily loading the clip unit 1 into the clip introducing device 200. The clip introducing device 200 and the clip unit 1 loaded in the clip introducing device 200 are also referred to as a clip device 300 (see FIG. 28).

### [Clip Introducing Device 200]

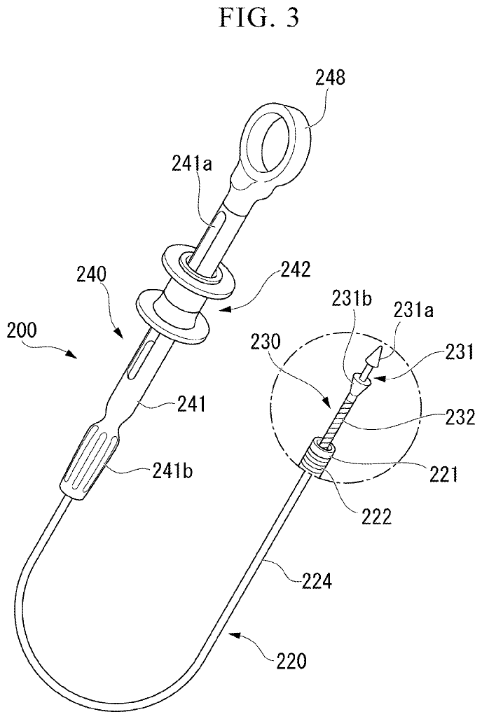

FIG. 3 is a perspective view of the clip introducing device 200.

The clip introducing device (applicator) 200 may include a sheath 220, an operating wire 230, and an operating portion 240. For example, the clip introducing device 200 can be inserted into a treatment instrument insertion channel of an endoscope and can be used in combination with the endoscope. Thus, the sheath 220 may be formed to be sufficiently larger than the length of the treatment instrument insertion channel of the endoscope. The sheath 220 may be flexible and may be curved to match the curve of an insertion portion of the endoscope.

The sheath 220 may include a distal tip 221, a distal coil 222, and a proximal coil 224 and may be formed into an elongated tubular shape as a whole. The distal coil 222 may be disposed on the distal end side of the sheath 220. The distal tip 221 may be disposed at the distal end of the distal coil 222.

As shown in FIG. 3, the operating wire (power transmission portion) 230 may include an arrowhead hook portion (connection portion, connector) 231 connected to the clip unit 1 and a wire 232 which operates the arrowhead hook portion 231.

The arrowhead hook portion 231 may include a substantially conical engagement portion 231a which engages with the clip unit 1 and a wire connection portion 231b provided at the proximal end of the engagement portion 231a. The arrowhead hook portion 231 may be formed of, for example, a metal material such as stainless steel.

The wire 232 can be inserted through the sheath 220 to be advanceable and retractable. The distal end of the wire 232 may be fixed to the proximal end of the wire connection portion 231b by, for example, welding.

As shown in FIG. 3, the operating portion 240 may include an operating portion main body 241, a slider 242, and a thumb ring 248. The operating portion main body 241, the slider 242, and the thumb ring 248 may be injection-molded from, for example, a resin material. The operating portion main body 241 may include a slit portion 241a and a rotating grip 241b on the distal end side. The slit portion 241a may support the slider 242 to be advanceable and retractable.

The slider 242 may be attached to be advanceable and retractable in the longitudinal direction of the operating portion main body 241 and the proximal end of the wire 232 may be attached thereto. As the slider 242 advances and retracts along the operating portion main body 241, the wire 232 can advance and retract with respect to the sheath 220 and the arrowhead hook portion 231 can advance and retract.

The thumb ring 248 may be attached to the proximal end of the operating portion main body 241 to be rotatable around the longitudinal axis of the operating portion main body 241.

### [Clip Unit 1]

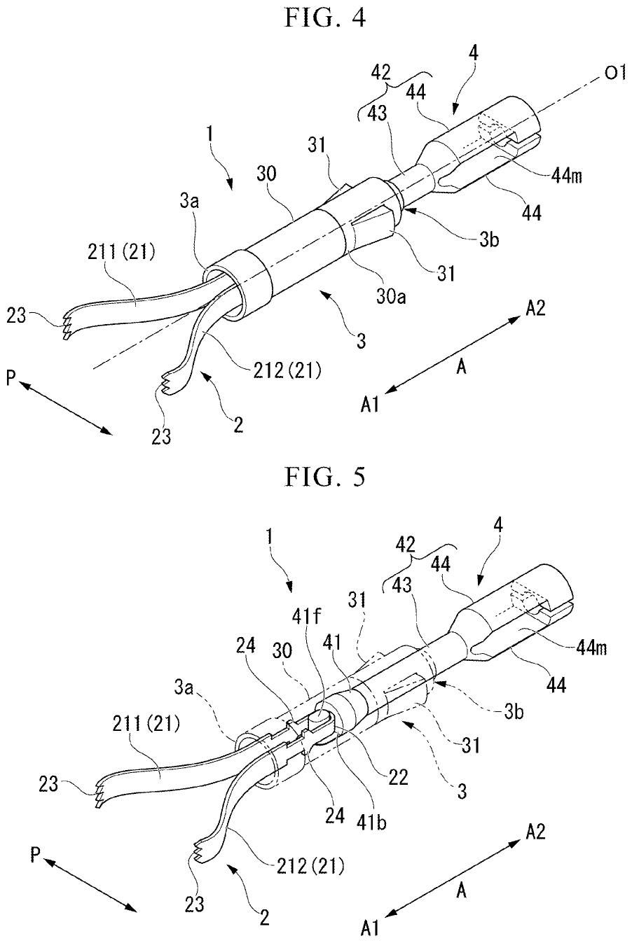

FIG. 4 is a perspective view of the clip unit 1 of the cartridge system 100. FIG. 5 is a perspective view of the clip unit 1 with the pressing tube 3 transparently displayed. The clip unit 1 may include a clip 2, a pressing tube 3 as a tightening member, and a connection member 4. In the following description, the side of the clip 2 in the longitudinal direction A of the clip unit 1 is defined as the distal end side (distal side) A1 of the clip unit and the side of the connection member 4 is defined as the proximal end side (proximal side) A2 of the clip unit 1.

The clip 2 may be, for example, formed by bending a metal plate material, such as a plate spring material formed of stainless steel or the like, at the center. The clip 2 may include a pair of arms 21 which can be opened and closed and a connection portion 22 connecting the pair of arms 21.

The pair of arms 21 may include a first arm 211 and a second arm 212. The first arm 211 and the second arm 212 may be symmetrically arranged with respect to a center axis O1 in the longitudinal direction A of the clip unit 1. The distal ends of the pair of arms 21 may be provided with tissue gripping portions 23 which face each other. The tissue gripping portions 23 may be formed by bending the distal ends of the pair of arms 21 inward.

The proximal ends of the pair of arms 21 may be provided with an engagement portion 24 which protrudes in the direction perpendicular to the center axis O1. An acute slope may be formed on the side of the tissue gripping portion 23 of the engagement portion 24 and an obtuse slope may be formed on the side of the connection portion 22 of the engagement portion 24.

The connection portion 22 may be bent into a U-shape and connected to the connection member 4. The connection portion 22 may be biased so that the pair of arms 21 are in an open state. Thus, the pair of arms 21 of the clip 2 may have a self-expanding force in an opening or closing direction P.

The pressing tube 3 may include a pressing tube main body 30 formed in a cylindrical shape and a protruding or retracting wing 31. The pressing tube main body 30 may be formed by injection-molding a highly rigid resin material. Further, the pressing tube main body 30 may be formed of metal instead of a highly rigid resin material. The protruding or retracting wing 31 may be formed by injection-molding a material softer than the clip 2, for example, a highly rigid resin material having appropriate elasticity such as PPA (polyphthalamide) and PA (polyamide).

The protruding or retracting wings 31 may be a pair of convex portions which protrude and retract with respect to an outer peripheral surface 30a of the pressing tube main body 30. The protruding or retracting wings 31 may be provided on both sides with the center axis O1 interposed therebetween. The protruding or retracting wing 31 can take a protruding posture to protrude radially outward with respect to the outer peripheral surface 30a. The protruding or retracting wing 31 can enter a retracted state to be retracted with respect to the outer peripheral surface 30a by receiving a force directed from the outside toward the inside in the radial direction. When the above-described force is released, the protruding or retracting wing 31 can return from the retracted state to the protruding state.

The connection member 4 can be connected to the connection portion 22 of the clip 2. The connection member 4 can be connected to the arrowhead hook portion 231 penetrating the sheath 220. That is, the connection member 4 can connect the clip 2 and the arrowhead hook portion 231. The connection member 4 may include an insertion portion 41 inserted into the internal space of the pressing tube 3 and a connection portion 42 provided at the proximal end of the insertion portion 41.

The insertion portion 41 may be an engagement portion which is engaged (e.g., connected or coupled) to the connection portion 22 of the clip 2. The insertion portion 41 may include a hook 41f provided on a distal end side A1 and a breaking portion 41b provided on a proximal end side A2 of the hook 41f. The hook 41f may be a hook which extends in the direction perpendicular to the center axis O1 and can be formed in a substantially cylindrical rod shape. The connection portion 22 of the clip 2 can be hooked onto the hook 41f.

The breaking portion 41b can break when the connection portion 22 is pulled toward the proximal end side A2 and a tensile breaking force of, for example, 20 N (Newton) to 90 N is applied to the hook 41f. Further, the breaking portion 41b may have a mechanism for releasing the connection between the connection portion 22 of the clip 2 and the hook 41f of the connection member 4. For example, the breaking portion 41b may be a mechanism that deforms (plastically deforms or elastically deforms) without breaking to release the connection between the connection portion 22 and the hook 41f.

The connection portion 42 may be an engagement portion to which the arrowhead hook portion 231 of the clip introducing device 200 can be engaged (connected). The connection portion 42 may include a connection portion main body 43 and a connection arm 44.

The connection arm 44 may be provided at the proximal end of the connection portion main body 43 and may be bifurcated. The connection arm 44 may be elastically deformable with respect to the connection portion main body 43 and can be opened and closed with respect to the connection portion main body 43. A notch portion 44m for gripping and accommodating the engagement portion 231a of the arrowhead hook portion 231 may be formed between the connection arms 44. The notch portion 44m may be formed in a shape that closely fits the outer peripheral surface of the engagement portion 231a of the arrowhead hook portion 231.

### [Cartridge 5]

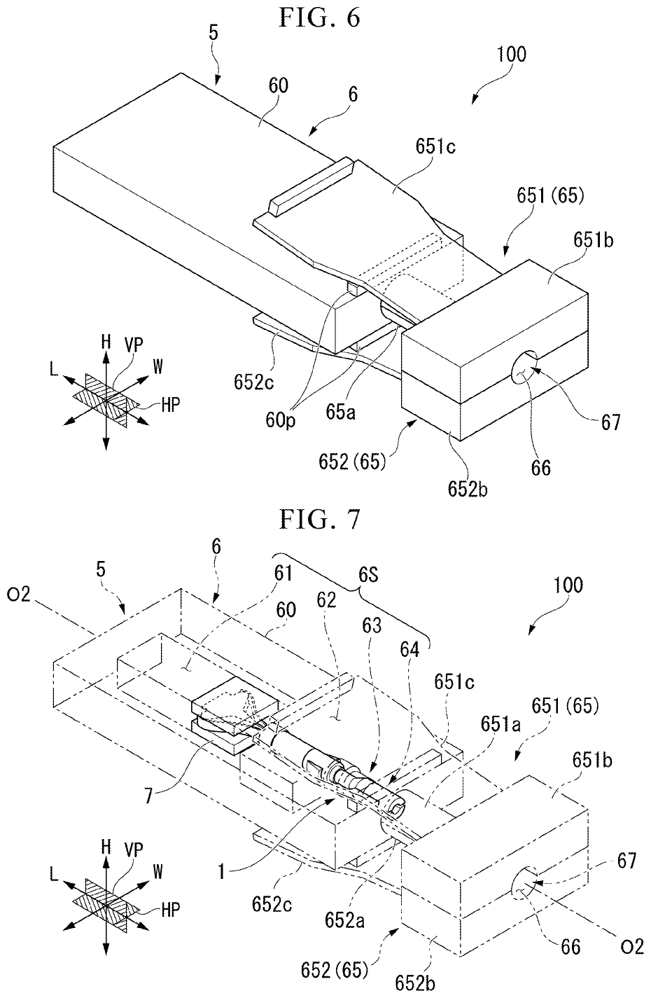

FIGS. 6 and 7 are perspective views of the cartridge 5.

The cartridge 5 may include a casing (a storing body) 6 and a regulation member 7. The cartridge 5 may have a width of about 10 mm to 20 mm, a length of about 50 mm, and a thickness of about 5 mm and may be formed in a size that is easy to hold in the hand.

One of two directions perpendicular to the longitudinal direction L of the cartridge 5 and perpendicular to each other is defined as the “width direction W” and the other is defined as the “height direction H”. Further, a plane horizontal to the longitudinal direction L and the width direction W is defined as a “horizontal plane HP”. A plane horizontal to the longitudinal direction L and the height direction H is defined as a “vertical plane VP”. Further, in the cartridge 5 storing the clip unit 1, the side of the pair of arms 21 is defined as the distal end side of the cartridge 5 and the side of the connection member 4 is defined as the proximal end side of the cartridge 5.

The casing 6 may include a casing main body 60, a compressing portion (e.g., a fixing member, a fixing body or other fixing means) 65, and a sheath connection portion 66. The fixing body includes surfaces forming a hole. The casing 6 may be, for example, manufactured by injection-molding a transparent resin material with appropriate hardness, such as ABS, PC, PP, PS, acrylic, and cycloolefin polymer. Since the casing 6 can be formed using a transparent resin material, the user can easily determine whether or not the clip unit 1 exists inside the casing.

The casing main body 60 may be formed into a rectangular box shape. The length of the casing main body 60 in the width direction W may be larger than the length of the casing main body 60 in the height direction H. The proximal end of the casing main body 60 may be provided with a pair of convex portions 60p which may protrude from both sides in the height direction H.

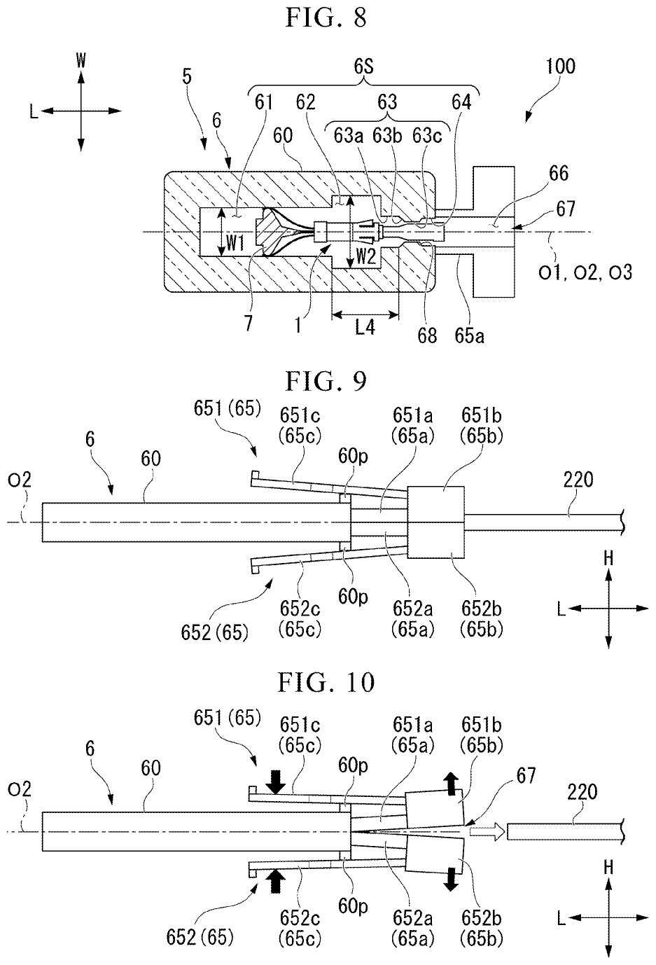

FIG. 8 is a cross-sectional view of the cartridge 5.

The casing main body 60 may be provided with a storage region 6S in which the clip unit 1 can be stored to be movable in the longitudinal direction L. The storage region 6S may include a first region (a container) 61, a second region 62, a folding region 63, and a sheath insertion region 64. As shown in FIG. 8, the first region 61, the second region 62, the folding region 63, and the sheath insertion region 64 may be arranged from the distal end toward the proximal end in the longitudinal direction L of the casing 6. The first region 61, the second region 62, the folding region 63, and the sheath insertion region 64 can be internal spaces formed symmetrically with respect to the vertical plane VP including a center axis O2 in the longitudinal direction L of the storage region 6S.

As shown in FIG. 8, the clip unit 1 can be stored in the storage region 6S with the center axis O1 of the clip unit 1 along the longitudinal direction L of the casing 6. The clip unit 1 can be stored in the storage region 6S with the opening or closing direction P of the pair of arms 21 aligned with the width direction W of the casing 6.

The first region 61 may be an internal space in which the clip unit 1 is stored to be movable in the longitudinal direction L. The first region 61 can communicate with, contact, connect with, or the like the second region 62.

The second region 62 may be an internal space in which the clip unit 1 is stored to be movable in the longitudinal direction L. The length of the second region 62 in the longitudinal direction L may be smaller than the length of the first region 61 in the longitudinal direction L. The second region 62 can communicate with, contact, connect with, or the like the folding region 63.

As shown in FIG. 8, the length W1 of the first region 61 in the width direction W may be smaller than the opening width W3 of the pair of arms 21 in the open state (see FIG. 23). The length W2 of the second region 62 in the width direction W (see FIG. 23) may be larger than the opening width W3 of the pair of arms 21 in the open state.

The folding region 63 may include an enlarged diameter portion 63a, a tapered portion 63b, and a reduced diameter portion 63c. The enlarged diameter portion 63a, the tapered portion 63b, and the reduced diameter portion 63c may be arranged from the distal end toward the proximal end.

The enlarged diameter portion 63a may be a region that allows the connection arm 44 of the connection member 4 to be elastically expanded (opened and closed). The enlarged diameter portion 63a allows the connection arm 44 of the connection member 4 to be opened and closed in the direction perpendicular to the center axis O1 when the arrowhead hook portion 231 of the clip introducing device 200 engages with the connection member 4 of the clip unit 1.

The tapered portion 63b may be provided on the proximal end side of the enlarged diameter portion 63a and is formed in a tapered shape. The diameter of the tapered portion 63b can increase from the proximal end side toward the distal end side. Therefore, when the pressing tube 3 is slid or moved from the distal end side toward the proximal end side, the protruding or retracting wing 31 of the pressing tube 3 can be accommodated, located, or stored inside the pressing tube main body 30.

The reduced diameter portion 63c may be a region which holds the protruding or retracting wing 31 in a retracted state. The reduced diameter portion 63c can hold the connection arm 44 of the connection member 4 while preventing the connection arm 44 from expanding when the clip unit 1 is accommodated in the cartridge 5.

When the pressing tube 3 is slid or otherwise moved along the tapered portion 63b from the distal end side toward the proximal end side, the protruding or retracting wing 31 of the pressing tube 3 can be accommodated, located, or stored in the pressing tube 3. Therefore, the reduced diameter portion 63c smoothly connected to the tapered portion 63b can hold the protruding or retracting wing 31 of the pressing tube 3 in an accommodated state.

The sheath insertion region 64 may be a region into which the distal end of the sheath 220 having passed through the sheath connection portion 66 can be inserted. The sheath insertion region 64 may be located on the proximal end side of the folding region 63 and may communicate with, contact, connect with, or the like the folding region 63. In the portion in which the sheath insertion region 64 and the folding region 63 are connected to each other, the length of the sheath insertion region 64 in the width direction W may be larger than the length of the folding region 63 in the width direction W. A sheath contact portion 68 may be formed in the sheath insertion region 64 to be able to come into contact with the distal tip 221 of the entered sheath 220.

FIGS. 9 and 10 are diagrams showing a compressing portion 65.

The compressing portion (e.g., a fixing member or other fixing means) 65 may be a member provided at the proximal end of the casing main body 60. The compressing portion 65 may include a first compressing portion 651 and a second compressing portion 652. The first compressing portion 651 and the second compressing portion 652 may be provided or arranged to face each other in the height direction H with the sheath connection portion 66 interposed therebetween.

The first compressing portion 651 may include a first connection portion 651a, a first sheath compressing portion 651b, and a first holding wing 651c. The first connection portion 651a can connect the casing main body 60 and the first sheath compressing portion 651b. The first sheath compressing portion 651b may be a portion which compresses the sheath 220. The first holding wing 651c may be a wing-shaped member that extends from the first sheath compressing portion 651b toward the distal end side.

The second compressing portion 652 may include a second connection portion 652a, a second sheath compressing portion 652b, and a second holding wing 652c. The second connection portion 652a can connect the casing main body 60 and the second sheath compressing portion 652b. The second sheath compressing portion 652b may be a portion which compresses the sheath 220. The second holding wing 652c may be a wing-shaped member that extends from the second sheath compressing portion 652b toward the distal end side.

In the following description, the first connection portion 651a and the second connection portion 652a are also referred to as “(a pair of) connection portions 65a”. The first sheath compressing portion 651b and the second sheath compressing portion 652b are also referred to as “(a pair of) sheath compressing portions 65b”. Further, the first holding wing 651c and the second holding wing 652c are also referred to as “(a pair of) holding wings 65c”.

The sheath connection portion 66 may be a groove into which the sheath 220 can be inserted. The sheath connection portion 66 may be an arcuate groove formed on the inner surfaces of the connection portion 65a and the sheath compressing portion 65b. The sheath connection portion 66 can communicate with, contact, connect with, or the like the sheath insertion region 64 of the storage region 6S. The sheath 220 can be inserted from a sheath insertion port (a hole) 67 provided at the proximal end of the sheath connection portion 66, can enter the sheath insertion region 64 through the sheath connection portion 66, and can face the sheath contact portion 68 of the sheath insertion region 64.

As shown in FIG. 10, when the pair of holding wings 65c are moved in the direction closer to the center axis O2, the pair of holding wings 65c can move the pair of sheath compressing portions 65b in the direction away from the center axis O2 using the pair of convex portions 60p as fulcrums to expand the sheath insertion port 67. As a result, the sheath 220 can be inserted into and removed from the sheath connection portion 66.

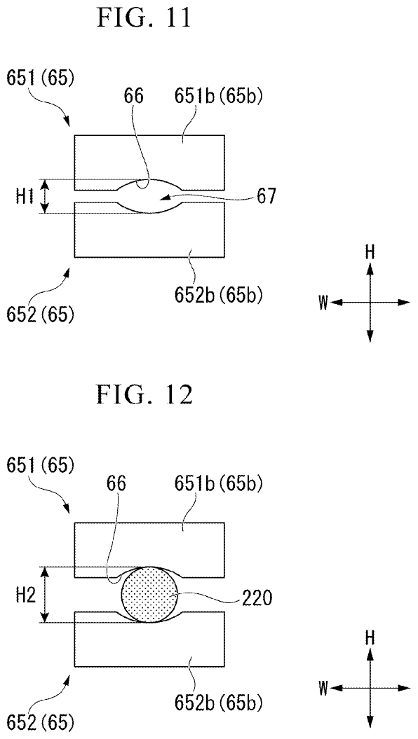

FIGS. 11 and 12 are diagrams showing the sheath connection portion 66.

The height H1 of the internal space of the sheath connection portion 66 may be smaller than the outer diameter H2 of the sheath 220. Thus, when the sheath 220 is inserted into the sheath connection portion 66, the sheath 220 can be sandwiched or located between the pair of sheath compressing portions 65b not to come off from the sheath connection portion 66 unless an external force is applied to the pair of holding wings 65c. That is, the sheath 220 inserted into the cartridge 5 remains connected to the cartridge 5 not to come off from the cartridge 5 unless an external force is applied to the compressing portion (fixing means) 65.

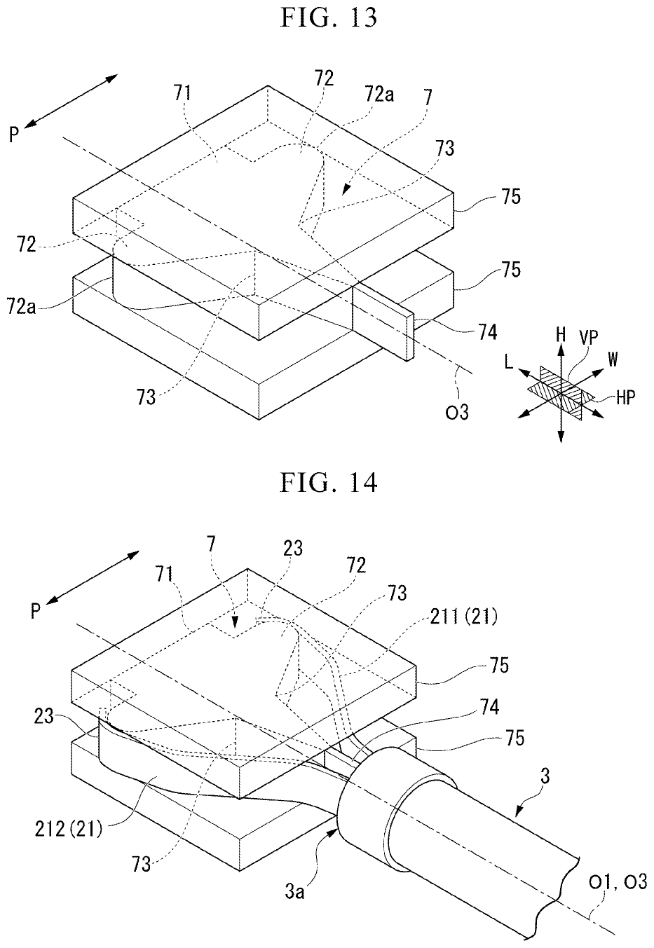

FIG. 13 is a perspective view of the regulation member 7.

The regulation member 7 may be stored to be movable toward the first region 61 and the second region 62 together with the clip unit 1. The regulation member 7 may be formed of, for example, resin like the casing 6. The regulation member 7 does not have to be formed of transparent resin such as the casing 6.

As shown in FIG. 13, the regulation member 7 may be formed in a symmetrical shape with respect to the vertical plane VP including a center axis O3. The regulation member 7 may include a distal end portion 71, a protruding portion 72, a tapered portion 73, and a pressing portion 74. The distal end portion 71, the protruding portion 72, the tapered portion 73, and the pressing portion 74 may be arranged from the distal end toward the proximal end along the direction of the center axis O3 of the regulation member 7.

FIG. 14 is a perspective view of the regulation member 7 gripped by the pair of arms 21.

The regulation member 7 can be gripped by the clip 2 with the center axis O3 substantially aligned with the center axis O1 of the clip unit 1. The clip unit 1 can be stored in the storage region 6S while the clip 2 grips the regulation member 7 as shown in FIG. 8. In such an example, the center axis O1, the center axis O2, and the center axis O3 may be aligned with one another.

The distal end portion 71 may be provided to protrude from the protruding portion 72 toward the distal end side. The distal end of the distal end portion 71 may be located at a position closer to the distal end than the tissue gripping portion 23 of the clip 2 for gripping the regulation member 7. Thus, the distal end portion 71 can prevent the tissue gripping portion 23 of the clip 2 from contacting the casing main body 60. Further, the distal end of the distal end portion 71 may be formed on a plane perpendicular to the center axis O3.

The protruding portion 72 may be a member that protrudes in the direction perpendicular to the center axis O3 (hereinafter, referred to as “protruding direction P”). The protruding portion 72 may be provided on both sides with the center axis O3 interposed or located therebetween. The protruding portion 72 may be gripped by the first arm 211 and the second arm 212. The opening or closing direction P of the pair of arms 21 gripping the protruding portion 72 may be substantially aligned with the protruding direction P of the protruding portion 72. The curvature of the outer peripheral surface of the protruding portion 72 may be smaller than the curvature of the inner peripheral surface of the tissue gripping portion 23. Thus, the pair of arms 21 can reliably grip the protruding portion 72.

The tapered portion 73 may be a member formed in a tapered shape. The tapered portion 73 may be provided on both sides with the center axis O3 interposed or located therebetween. The tapered portion 73 may have a smaller length in the protruding direction P than the protruding portion 72. The length of the tapered portion 73 in the protruding direction P can become smaller from the distal end side toward the proximal end side.

The pressing portion 74 may be a plate-shaped member that can regulate the minimum approach distance between the clip 2 and the pressing tube 3. The pressing portion 74 may be provided on the proximal end side of the tapered portion 73. Since the pressing portion 74 engages with the edge of the distal end opening 3a, the pressing portion cannot enter the internal space of the pressing tube 3 from the distal end opening 3a. Therefore, even when the clip 2 is pulled in the direction closer to the pressing tube 3, the pressing portion 74 can regulate the minimum approach distance between the clip 2 and the pressing tube 3 by engaging with the edge of the distal end opening 3a.

As shown in FIG. 13, the regulation member 7 can be sandwiched or located between auxiliary members 75 from the height direction H. The auxiliary member 75 can adjust the position of the regulation member 7 in the height direction H so that the regulation member 7 is gripped by the pair of arms 21. When there is no need to adjust the position of the regulation member 7 in the height direction, the auxiliary member 75 may not be necessary.

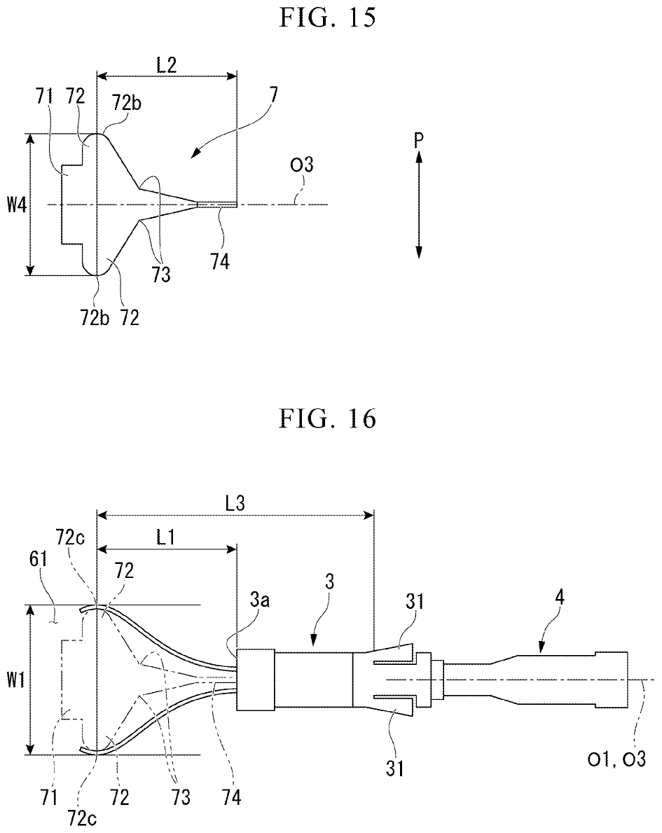

FIG. 15 is a plan view of the regulation member 7.

In the protruding portion 72, a portion that protrudes most in the protruding direction P from the center axis O3 is defined as a maximum protruding point 72b. The length W4 between the maximum protruding points 72b may be slightly smaller than the length W1 of the first region 61 in the width direction W. The length from the maximum protruding point 72b to the proximal end of the pressing portion 74 in the direction of the center axis O3 is defined as L2.

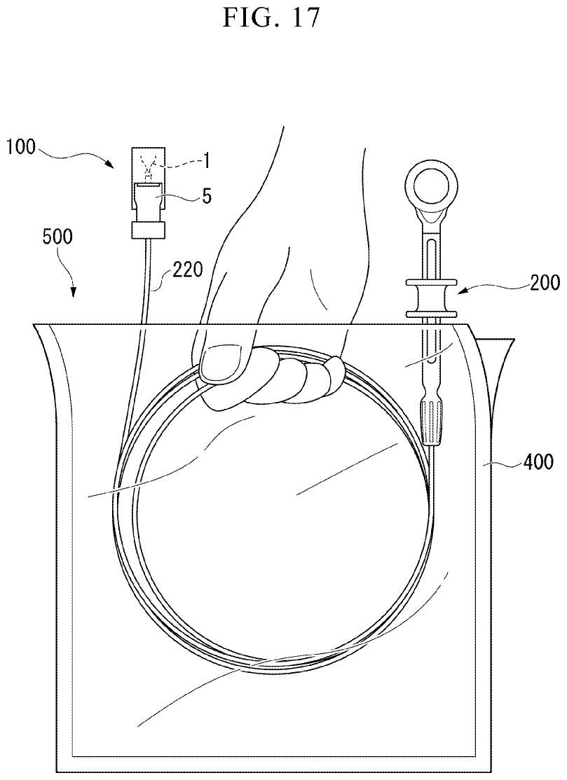

FIG. 16 is a plan view of the regulation member 7 gripped in the first region 61.

The regulation member 7 may be stored in the first region 61 while being gripped by the pair of arms 21. The length W1 of the first region 61 in the width direction W may be smaller than the opening width W3 of the pair of arms 21 in an open state. Thus, the pair of arms 21 grip the regulation member 7 in a closed state from an open state. The opening width of the pair of arms 21 may be approximately the length W1 of the first region 61 in the width direction W. The pair of arms 21 can contact the casing main body 60 in the opening or closing direction P. The length in the direction of the center axis O3 from the contact point 72c between the pair of arms 21 and the casing main body 60 to the proximal end of the pressing portion 74 is defined as L1.

The length L1 in the direction of the center axis O3 from the contact point 72c to the proximal end of the pressing portion 74 may be equal to or larger than the length L2 in the direction of the center axis O3 from the maximum protruding point 72b to the proximal end of the pressing portion 74. In such an example, the clip 2 can be reliably engaged with the regulation member 7 and can be pulled toward the proximal end side when the clip 2 is pulled toward the proximal end side.

As shown in FIGS. 15 and 16, the length W4 between the maximum protruding points 72b may be slightly smaller than the length W1 of the first region 61 in the width direction W. Thus, the regulation member 7 can be held in a gripped state by the pair of arms 21 in the first region 61.

The length L3 (see FIG. 16) in the direction of the center axes O1 and O3 from the contact point 72c to the distal end of the protruding or retracting wing 31 may be equal to or larger than the distance L4 (see FIG. 8) from the distal end of the second region 62 to the proximal end of the tapered portion 63b.

### [Method of Loading Clip Unit 1]

Next, the operation of the clip system 500 will be described with reference to FIGS. 17 to 27. In FIGS. 17 to 27, gloves worn by the user on the hands are omitted.

FIG. 17 is a diagram showing the opened sterile pack 400.

The user may open the sterile pack 400 and take out the cartridge system 100 and the clip introducing device 200 from the sterile pack 400. The cartridge system 100 can be connected to the clip introducing device 200.

As shown in FIG. 2, when the cartridge system 100 is not connected to the clip introducing device 200, the user can expand the sheath insertion port 67 and insert the sheath 220 of the clip introducing device 200 into the sheath connection portion 66 as shown in FIG. 10.

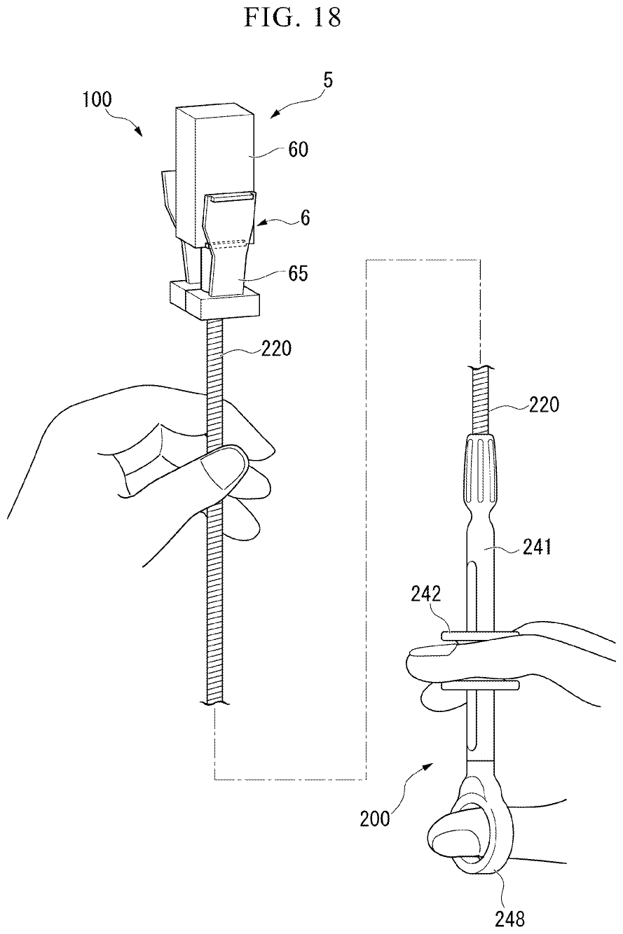

FIG. 18 is a diagram showing the clip introducing device 200 held by the user.

The user may hold the operating portion 240 of the clip introducing device 200 with a right hand and hold the sheath 220 with a left hand. The cartridge system 100 may be attached to the sheath 220 so as not to come off, be removed, become disconnected, or the like from the sheath 220. Thus, the user does not need to have the cartridge system 100.

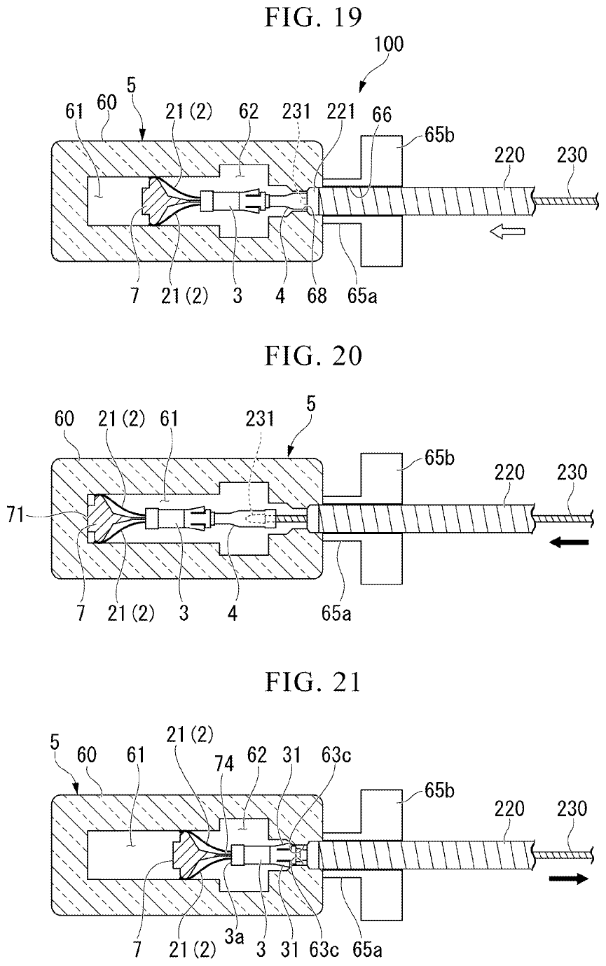

FIGS. 19 to 25 are diagrams illustrating a method of loading the clip unit 1 into the clip introducing device 200 using the cartridge 5. As shown in FIG. 19, the sheath 220 may be inserted into the sheath connection portion 66 and the distal tip 221 of the sheath 220 contacts the sheath contact portion 68. When the distal tip 221 of the sheath 220 does not contact the sheath contact portion 68, the user can press the sheath 220 toward the distal end side so that the distal tip 221 can contact the sheath contact portion 68.

As shown in FIG. 20, the user can operate the operating portion 240 to advance the operating wire 230 relative to the sheath 220 so that the arrowhead hook portion 231 can advance. The arrowhead hook portion 231 may be connected to the connection member 4 of the clip unit 1.

Further, when the arrowhead hook portion 231 is connected to the connection member 4 of the clip unit 1 in advance, the user does not have to carry out the procedure of advancing the arrowhead hook portion 231.

As shown in FIG. 21, the user can pull the operating wire 230. The clip 2 of the clip unit 1 may be pulled toward the proximal end side by the connection member 4 connected to the arrowhead hook portion 231. The hook 41f of the connection member 4 can pull the connection portion 22 of the clip 2 without breaking. The regulation member 7 can move in the first region 61 of the storage region 6S while contacting the pair of arms 21. Since the pair of arms 21 having a self-expanding force engage with the edge of the distal end opening 3a of the pressing tube 3, the pressing tube 3 may also pulled toward the proximal end side together with the clip 2.

The pair of arms 21 can contact the casing main body 60 in the opening or closing direction P in the first region 61. The clip 2 can be pulled into the internal space of the pressing tube 3 by the frictional force generated by the contact between the pair of arms 21 and the casing main body 60 and the clip 2 can be suitably prevented from being locked in the closed state by the pressing tube 3.

Since the pressing portion 74 of the regulation member 7 engages with the edge of the distal end opening 3a of the pressing tube 3 when the clip 2 is pulled toward the proximal end side, the minimum approach distance between the clip 2 and the pressing tube 3 can be regulated. The pressing portion 74 can contact the pressing tube 3 while contacting the pair of arms 21 by pulling the operating wire 230 and can regulate the relative movement of the regulation member 7 with respect to the pressing tube 3. Even in this configuration, it is possible to prevent the clip 2 pulled toward the proximal end side by the connection member 4 from being pulled into the internal space of the pressing tube 3 and being locked in the closed state by the pressing tube 3.

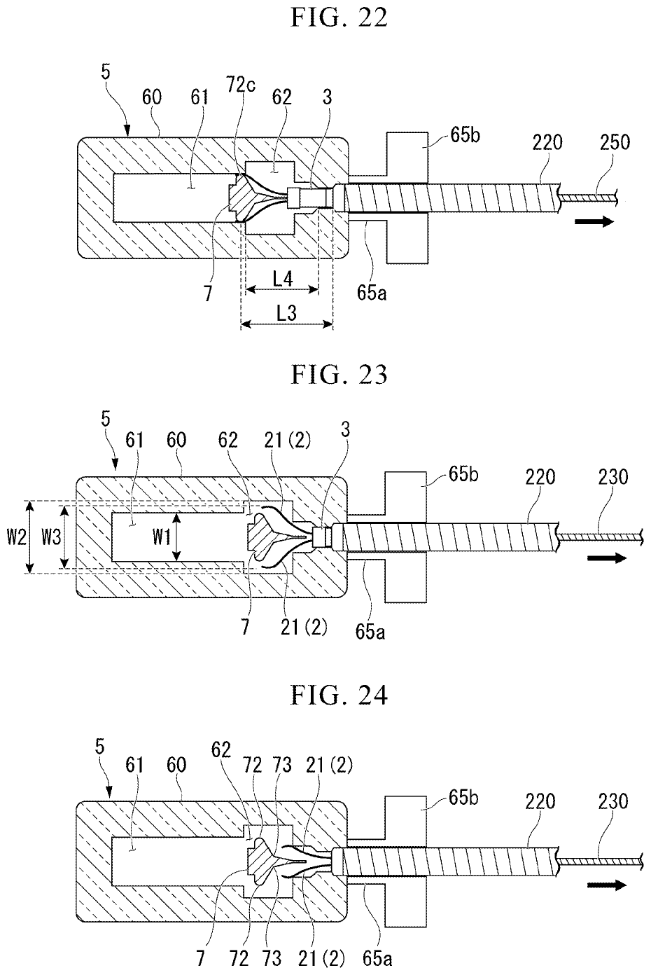

The user can further pull the clip unit 1 toward the proximal end side. As shown in FIG. 22, the pressing tube 3 can pass through the folding region 63. The pressing tube 3 can be slid or moved along the reduced diameter portion 63c from the distal end side toward the proximal end side and the protruding or retracting wing 31 of the pressing tube 3 can be accommodated or stored in the pressing tube main body 30. The pressing tube 3 in which the protruding or retracting wing 31 can be accommodated or stored inside the pressing tube main body 30 can be pulled into the sheath 220.

As shown in FIG. 22, the length L3 in the direction of the center axes O1 and O3 from the contact point 72c to the distal end of the protruding or retracting wing 31 may be equal to or larger than the distance L4 from the distal end of the second region 62 to the proximal end of the tapered portion 63b. Thus, when the protruding or retracting wing 31 of the pressing tube 3 is accommodated or stored inside the pressing tube 3, the contact point 72c between the pair of arms 21 and the casing main body 60 can be located in the first region 61. That is, the pair of arms 21 grip the regulation member 7 and are not pulled into the internal space of the pressing tube 3 until the protruding or retracting wing 31 is accommodated or stored inside the pressing tube 3.

As shown in FIG. 23, the user can pull the regulation member 7 to the second region 62 by further pulling the operating wire 230. The length W2 of the second region 62 in the width direction W may be larger than the opening width W3 of the pair of arms 21 in an open state. Thus, the regulation member 7 may not be gripped by the pair of arms 21 in the second region 62.

As shown in FIG. 24, the user can further pull the operating wire 230. The clip 2 can be separated from the regulation member 7 and can be pulled toward the proximal end side. In the regulation member 7, the tapered portion 73 can be formed on the proximal end side of the protruding portion 72 gripped by the pair of arms 21. Thus, the pair of arms 21 are less likely to be caught by the regulation member 7 when the regulation member 7 is pulled toward the proximal end side.

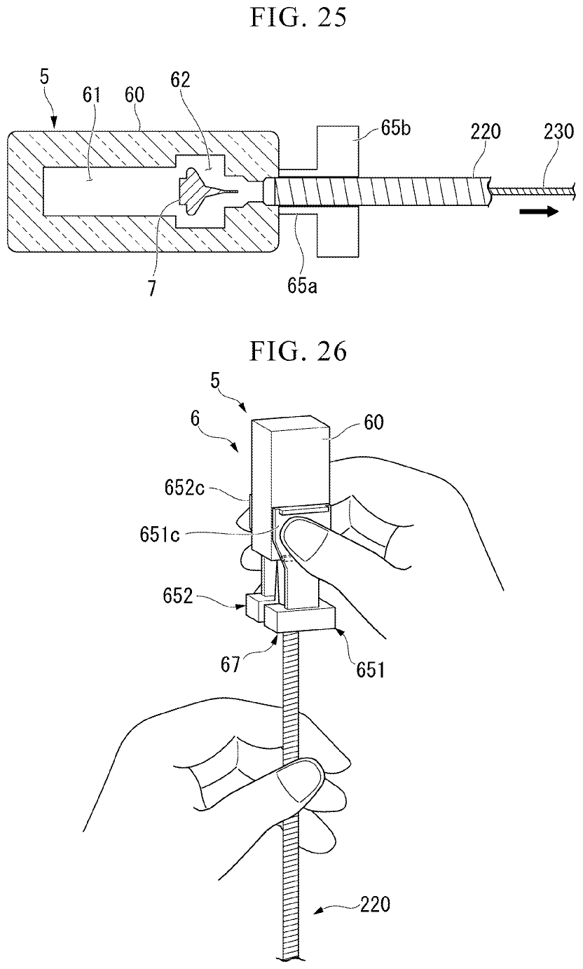

As shown in FIG. 25, the user may further pull the operating wire 230. Since the pair of arms 21 do not grip the regulation member 7, the minimum approach distance between the clip 2 and the pressing tube 3 may not be regulated. The clip 2 pulled toward the proximal end side can be pulled into the sheath 220 while being pulled into the internal space of the pressing tube 3. The clip 2 may be pulled into the sheath 220 while not being pulled into the internal space of the pressing tube 3. Accordingly, the loading of the clip unit 1 into the clip introducing device 200 is completed.

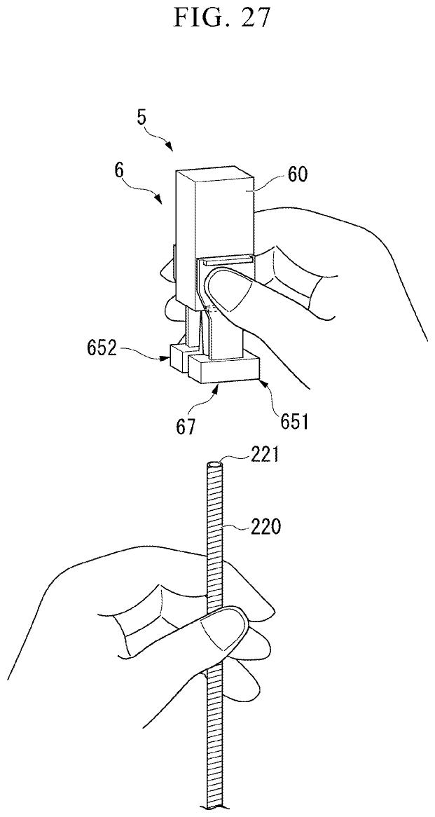

FIGS. 26 and 27 are diagrams showing the cartridge 5 from which the sheath 220 is pulled out.

The user can expand the sheath insertion port 67 and pull out the sheath 220 from the sheath connection portion 66 by gripping the pair of holding wings 65c and moving the holding wings in the direction closer to the center axis O2.

### [Operation and Action of Clip Unit 1]

Next, the operation and action of the clip unit 1 will be described with reference to FIGS. 28 to 32.

The connection member 4 of the loaded clip unit 1 may be connected to the arrowhead hook portion 231 penetrating the sheath 220. The protruding or retracting wing 31 can be pressed by the inner peripheral surface of the sheath 220 to be in a retracted state.

The pair of arms 21 of the loaded clip unit 1 can be pressed by the inner peripheral surface of the sheath 220 to be in a closed state. The engagement portion 24 may be located closer to the distal end side than a proximal end opening 3b and the pair of arms 21 may not be locked in a closed state.

FIG. 28 is a diagram showing the clip unit 1 introduced into the body.

The user can introduce the clip unit 1 loaded into the sheath 220 into the body through the channel of the endoscope. Next, the user can advance the slider 242 along the operating portion main body 241 so that the arrowhead hook portion 231 advances. The user can advance the clip unit 1 until the protruding or retracting wing 31 comes out of the sheath 220. The protruding or retracting wing 31 comes out of the sheath 220 and can return from the retracted state to the protruding state as a basic posture.

When the distal end side comes out of the sheath 220, the pair of arms 21 can return to the open state while the clip 2 moves toward the distal end side relative to the pressing tube 3 by using the self-expanding force of the pair of arms 21 as a restoring force. Even when the pair of arms 21 return to the open state and protrude the most from the pressing tube 3, the engagement portion 24 can be disposed in the inner region of the pressing tube 3.

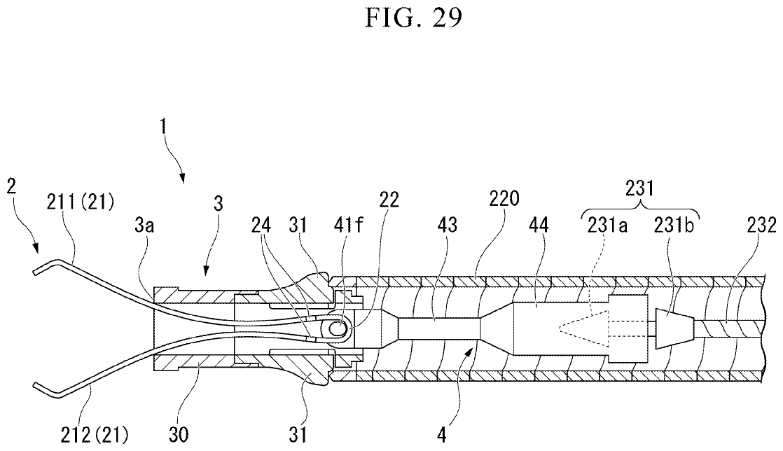

FIG. 29 is a diagram showing the clip unit 1 with the pair of arms 21 closed.

The user can retract the arrowhead hook portion 231 by retracting the slider 242 along the operating portion main body 241. The connection member 4 connected to the arrowhead hook portion 231 can pull the clip 2. The pair of arms 21 having a self-expanding force can be pulled toward the proximal end side to press out the distal end opening 3a of the pressing tube toward the proximal end side. Since the protruding or retracting wing 31 in the protruding state engages with the sheath 220, the protruding or retracting wing is not pulled into the sheath 220. Thus, the clip 2 pulled by the connection member 4 can be pulled into the pressing tube 3.

When the connection member 4 pulls the connection portion 22 of the clip 2 toward the proximal end side of the pressing tube 3, the pair of arms 21 can be pulled into the pressing tube 3 and the pair of arms 21 can be gradually closed. When the pulling force of the connection portion 22 is released in this state, the clip 2 can return to the open state while moving toward the distal end side by using the self-expanding force of the pair of arms 21 as a restoring force. The user can return the pair of arms 21 to the open state to regrasp the tissue.

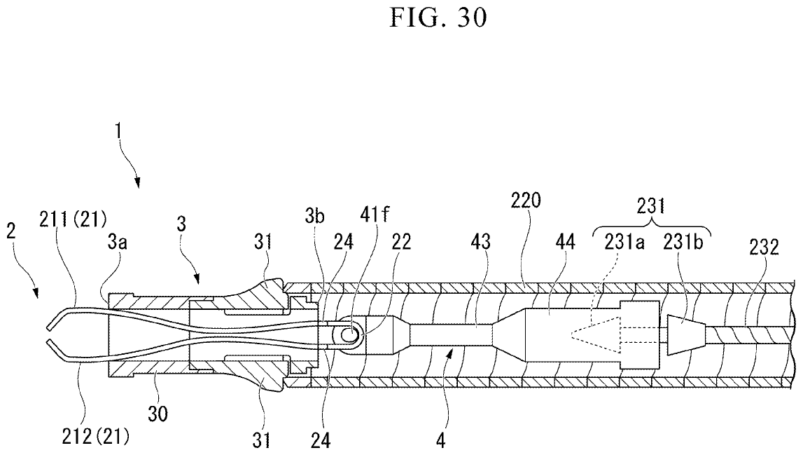

FIG. 30 is a diagram showing the clip unit 1 with the clip 2 locked.

When the connection portion 22 is further pulled toward the proximal end side of the pressing tube 3, the engagement portion 24 can be pulled from the proximal end opening 3b toward the proximal end side. Since an obtuse slope can be formed on the side of the connection portion 22 of the engagement portion 24, the engagement portion 24 can be easily pulled from the proximal end opening 3b toward the proximal end side. On the other hand, since an acute slope can be formed on the side of the tissue gripping portion 23 of the engagement portion 24, the engagement portion 24 and the proximal end opening 3b can engage with each other when the engagement portion 24 is pulled from the proximal end opening 3b to the proximal end side. As a result, the clip 2 can be regulated (e.g., hindered from moving toward the distal end side with respect to the pressing tube 3) and the pair of arms 21 can be locked in the closed state. When the pair of arms 21 are locked in the closed state, the pair of arms 21 cannot be returned to the open state.

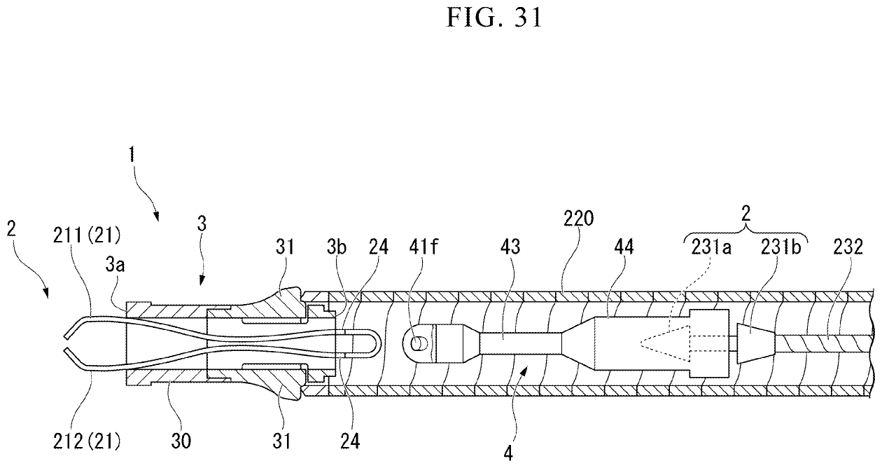

FIG. 31 is a diagram showing the clip unit 1 with the clip 2 separated.

The user can further pull the clip 2 and when a tensile breaking force of 20 N (Newton) to 90 N is applied to the hook 41f, and the breaking portion 41b can break. The breaking strength of the breaking portion 41b can be lower than the breaking strength of the connection portion main body 43. Thus, the breaking portion 41b can be broken instead of the connection portion main body. Stated differently, the breaking portion 41b can break while the connection portion main body 43 can remain intact.

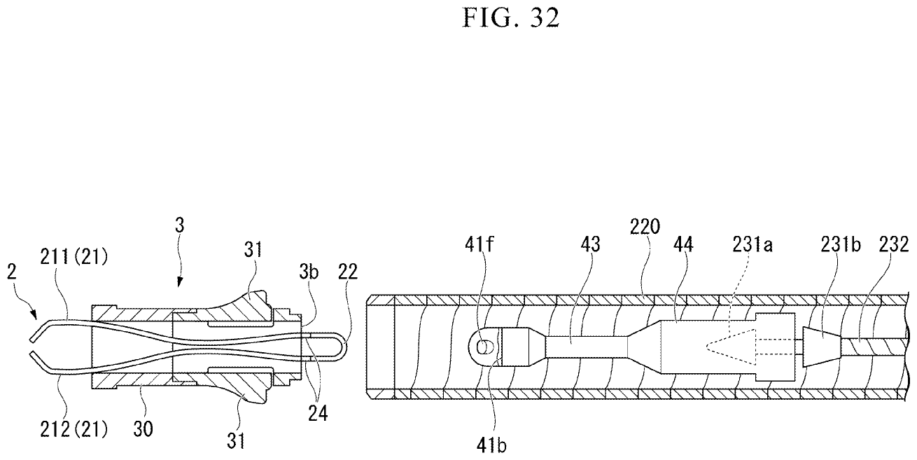

FIG. 32 is a diagram showing the clip unit 1 after breaking.

The user can retract the sheath 220 and leave the clip 2 with the tissue ligated in the body.

According to the clip system 500 of this embodiment, the cartridge system 100 and the applicator 200 can be accommodated in the same sterile pack 400 and the cartridge system 100 can be attached to the applicator 200 in advance. Thus, there is less work to load the clip unit 1 into the applicator 200 using the cartridge system 100 after opening the sterile pack, and the user can use the clip unit 1 loaded into the applicator 200 as soon as possible.

Although the first embodiment of the present disclosure has been described above in detail with reference to the drawings, the specific configuration is not limited to this embodiment, and design changes may be made within the scope of the present disclosure. Further, the components shown in the above-described embodiment and the modified examples shown below can be configured by appropriately combining them.

### Modified Example 1-1

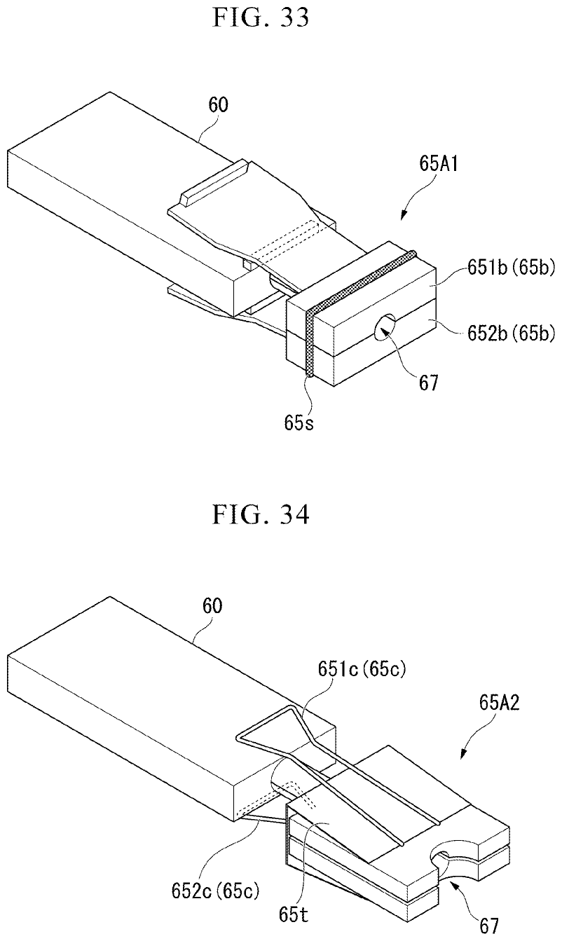

FIG. 33 is a diagram showing a compressing portion 65A1 which is a modified example of the compressing portion 65.

In the compressing portion 65A1, a rubber band 65s can be wrapped around the pair of sheath compressing portions 65b. The compressing portion 65A1 can more reliably prevent the sheath 220 inserted into the sheath connection portion 66 from coming off.

### Modified Example 1-2

FIG. 34 is a diagram showing a compressing portion 65A2 which is a modified example of the compressing portion 65.

The compressing portion 65A2 may include a metal plate 65t which can compress the pair of sheath compressing portions 65b in the height direction H. The metal plate 65t and the pair of holding wings 65c may have the same structure as a double clip. The user can reduce a force in which the metal plate 65t presses the pair of sheath compressing portions 65b by grasping the pair of holding wings 65c and moving the pair of holding wings in the direction closer to the center axis O2. The compressing portion 65A2 can more reliably prevent the sheath 220 inserted into the sheath connection portion 66 from coming off.

### Second Embodiment

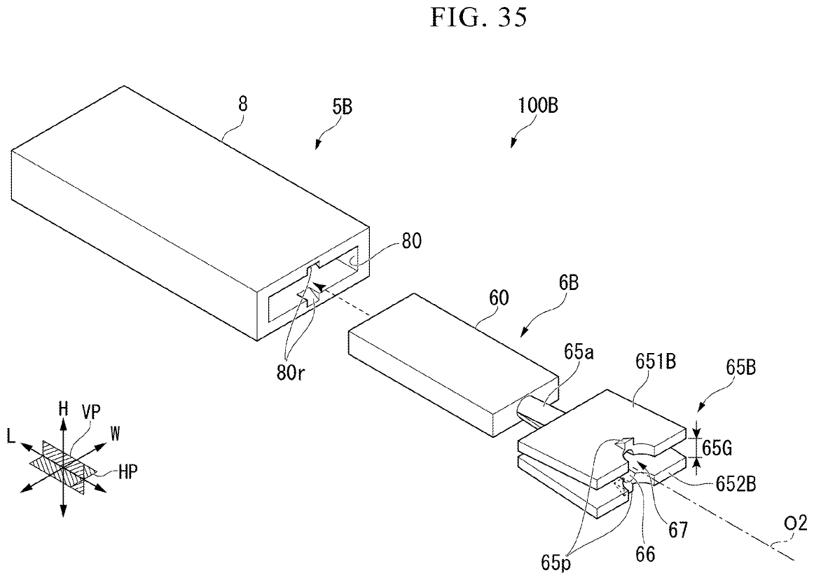

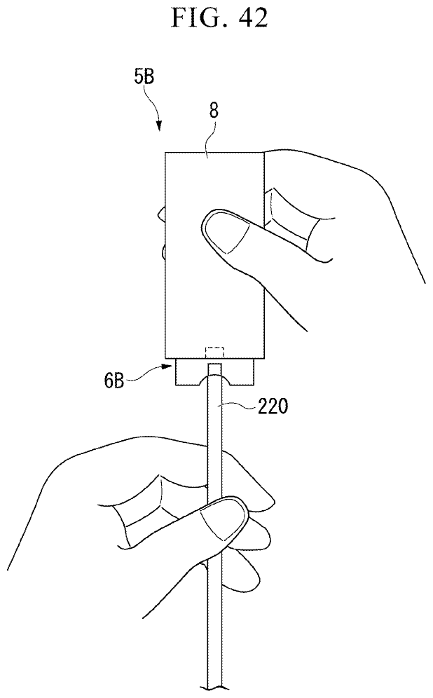

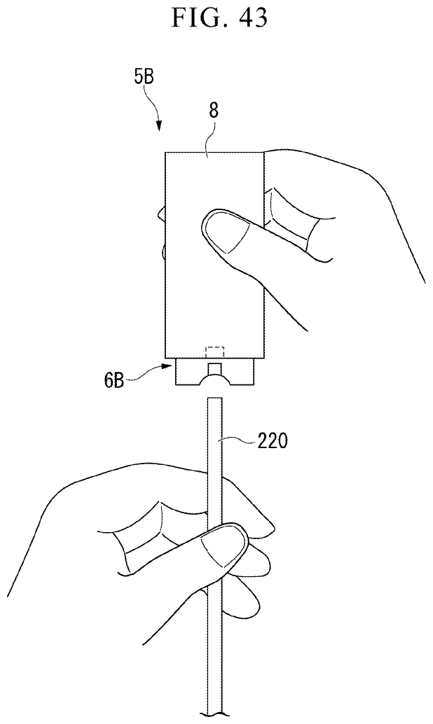

A second embodiment of the present disclosure will be described with reference to FIGS. 35 to 43. In the following description, components that are common to those already described will be given the same reference numerals and redundant description will be omitted.

FIG. 35 is a diagram showing a cartridge system 100B according to a second embodiment.

Similar to the cartridge system 100 of the first embodiment, the cartridge system 100B may be packaged in the sterile pack 400 as the clip system 500 together with the clip introducing device (applicator) 200. The cartridge system 100B may include the clip unit 1 and a cartridge 5B which can accommodate or store the clip unit 1.

The cartridge 5B may include a casing (inner casing) 6B, the regulation member 7, and an outer casing (or other fixing member or means) 8. The cartridge 5B can accommodate the clip unit 1.

Similar to the casing 6 of the first embodiment, the casing (the inner casing) 6B can store the clip unit 1 together with the regulation member 7. The casing 6B can be accommodated in the outer casing 8. The casing 6B may include the casing main body 60, a compressing portion 65B, and the sheath connection portion 66.

The compressing portion 65B may be a plate-shaped member provided at the proximal end of the casing main body 60. The compressing portion 65B may include a first compressing portion 651B and a second compressing portion 652B. The first compressing portion 651B and the second compressing portion 652B may be provided or arranged to face each other in the height direction H with the sheath connection portion 66 interposed therebetween.

The compressing portion 65B may include the connection portion 65a connected to the casing main body 60. The connection portion 65a can individually connect the first compressing portion 651B and the second compressing portion 652B to the casing main body 60. The connection portion 65a may be bent so that the first compressing portion 651B and the second compressing portion 652B can be separated from each other. Thus, a gap 65G can be formed between the first compressing portion 651B and the second compressing portion 652B. The first compressing portion 651B and the second compressing portion 652B can be further separated from each other on the proximal end side rather than the distal end side.

The compressing portion 65B may be provided with the pair of convex portions 65p which protrude from both sides in the height direction H. The pair of convex portions 65p may be provided on both sides with the sheath insertion port 67 interposed therebetween.

The outer casing (or other fixing member or means) 8 may be a casing which can accommodate the casing 6B. The casing 6B may be inserted into and removed from an insertion port 80 formed on the proximal end side of the outer casing 8. The insertion port 80 of the outer casing 8 may be provided with a pair of concave portions 80r which can engage with the pair of convex portions 65p of the accommodated casing 6B.

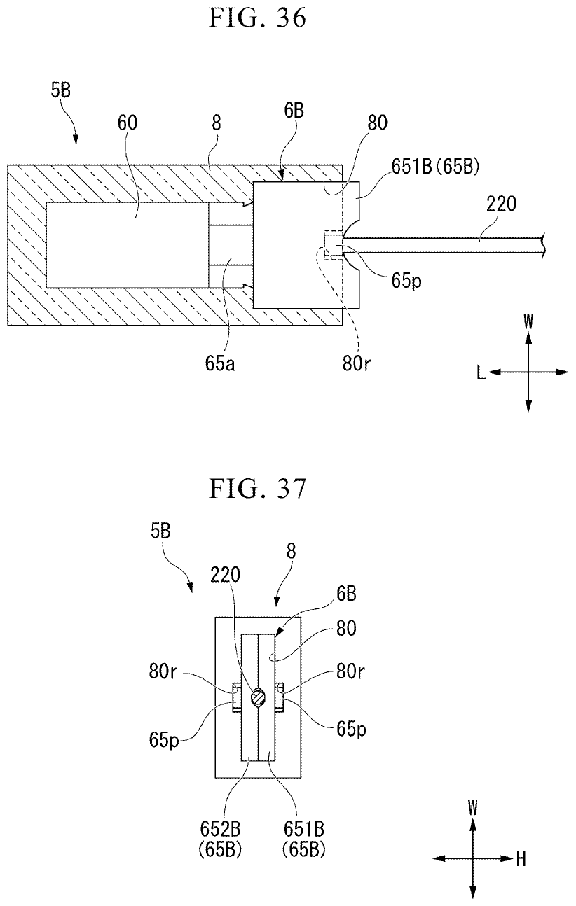

FIG. 36 is a diagram showing the casing 6B accommodated in the outer casing 8.

The casing 6B in which the sheath 220 can be inserted into the sheath connection portion 66 can be accommodated in the outer casing 8. When the casing 6B is accommodated in the outer casing 8, the pair of convex portions 65p can engage with the pair of concave portions 80r and the pair of concave portions 80r can move the compressing portion 65B including the pair of convex portions 65p in the direction closer to the center axis O2. Further, in an example in which the outer casing 8 moves the compressing portion 65B in the direction closer to the center axis O2 when the casing 6B is accommodated in the outer casing 8, the pair of convex portions 65p and the pair of concave portions 80r may not be provided.

FIG. 37 is a view of the cartridge 5B shown in FIG. 36 when viewed from the proximal end side.

When the casing 6B is accommodated in the outer casing 8, the sheath 220 can be sandwiched or located between the compressing portions 65B not to come off from the sheath connection portion 66. That is, when the casing 6B is accommodated in the outer casing (or other fixing member or fixing means, outer case) 8, the sheath 220 inserted into the cartridge 5B can remain connected to the cartridge 5B not to come off from the cartridge 5B.

The clip unit 1 may be loaded into the clip introducing device 200 by the same method as the first embodiment while the sheath 220 is inserted into the casing main body 60 of the cartridge 5B.

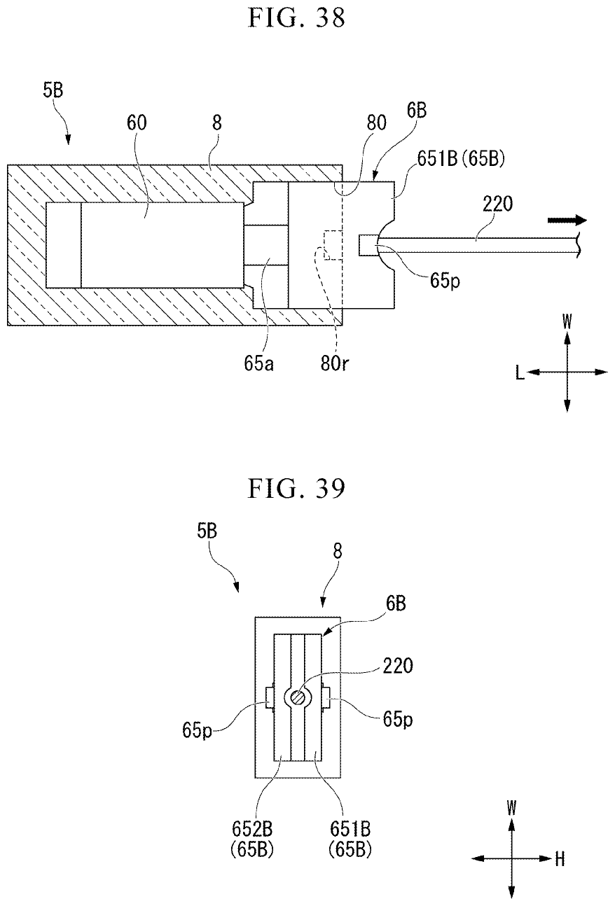

FIG. 38 is a diagram showing the casing 6B separated from the outer casing 8.

When the sheath 220 moves in the direction away from the cartridge 5B, the compressing portion 65B sandwiching the sheath 220 can move together with the sheath 220. As a result, the casing 6B can be pulled out from the outer casing 8 and the casing 6B and the outer casing 8 are separated.

FIG. 39 is a view of the cartridge 5B shown in FIG. 38 when viewed from the proximal end side.

When the casing 6B and the outer casing 8 are separated, the compressing portion 65B can move in the direction away from the center axis O2 by the elastic force of the compressing portion 65B so that the sheath insertion port 67 can be expanded. As a result, the sheath 220 can be inserted into and removed from the sheath connection portion 66.

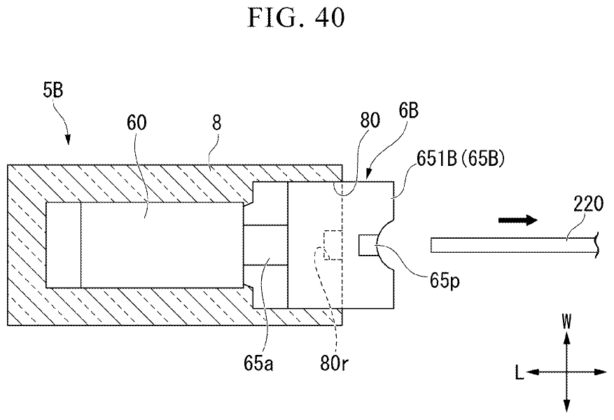

FIG. 40 is a diagram showing the casing 6B from which the sheath 220 is pulled out.

When the sheath 220 moves in the direction away from the cartridge 5B, the sheath 220 can come off, separate from, or the like from the sheath connection portion 66.

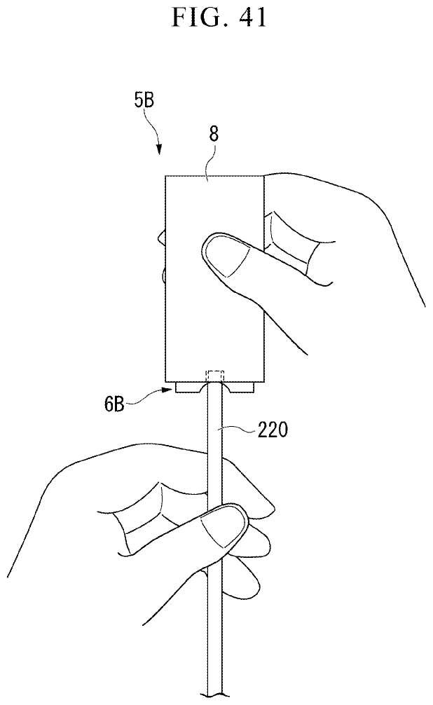

FIGS. 41 to 43 are diagrams illustrating the procedure of pulling out the sheath 220.

The user can separate the casing 6B from the outer casing 8 and pull out the sheath 220 from the casing 6B just by pulling out the sheath 220 from the cartridge 5B.

According to the clip system 500 of this embodiment, the cartridge system 100B and the applicator 200 can be accommodated in the same sterile pack 400 and the cartridge system 100B can be attached to the applicator 200 in advance. Thus, there is less work to load the clip unit 1 into the applicator 200 using the cartridge system 100B after opening the sterile pack, and the user can use the clip unit 1 loaded into the applicator 200 as soon as possible.

Although the second embodiment of the present disclosure has been described above in detail with reference to the drawings, the specific configuration is not limited to this embodiment, and design changes may be made within the scope of the present disclosure. Further, the components shown in the above-described embodiment and the modified examples shown below can be configured by appropriately combining them.

### Modified Example 2-1

In the above-described embodiment, the outer casing 8 may be a casing which can contain the entire casing 6B. However, the shape of the outer casing is not limited thereto. The outer casing may be any casing as long as at least the compressing portion 65B can be contained and the connection between the sheath 220 and the cartridge 5B can be maintained.

### Third Embodiment

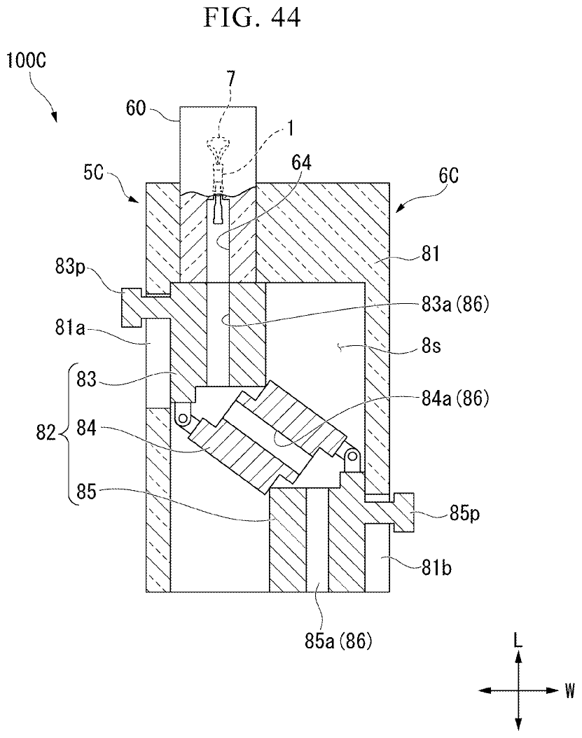

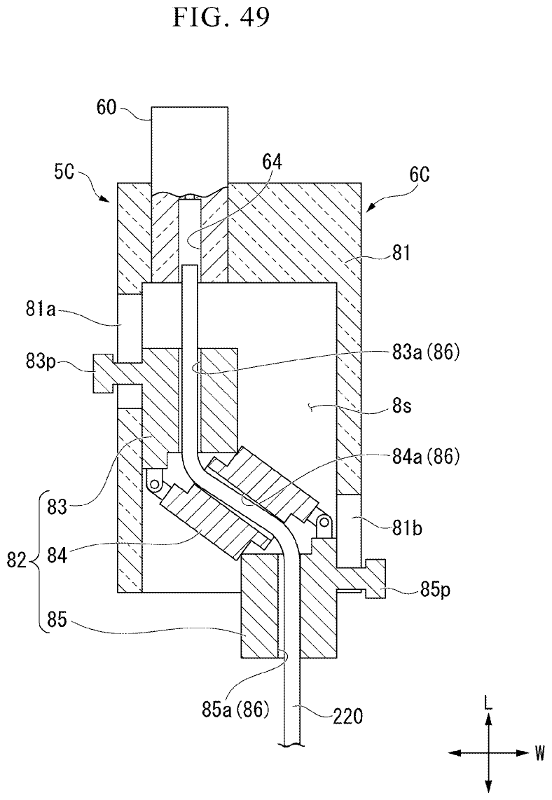

A third embodiment of the present disclosure will be described with reference to FIGS. 44 to 50. In the following description, components that are common to those already described will be given the same reference numerals and redundant description will be omitted.

FIG. 44 is a diagram showing a cartridge system 100C according to the third embodiment.

Similar to the cartridge system 100 of the first embodiment, the cartridge system 100C can be packaged in the sterile pack 400 as the clip system 500 together with the clip introducing device (applicator) 200. The cartridge system 100C may include the clip unit 1 and a cartridge 5C which can accommodate the clip unit 1.

The cartridge 5C may include a casing 6C and a regulation member 7. The cartridge 5C may accommodate the clip unit 1.

The casing 6C can store the clip unit 1 together with the regulation member 7 similar to the casing 6 of the first embodiment. The casing 6C may include a casing main body 60 and a casing connection portion (fixing means) 81.

The casing connection portion 81 can connect the sheath 220 to the casing main body 60. The casing connection portion 81 may be formed in a substantially box shape and may support the casing main body 60 so that the sheath insertion region 64 of the casing main body 60 can communicate with, connect with, contact, engage, or the like the internal space 8s. The casing connection portion 81 may include a casing movable portion 82 in the internal space 8s.

The casing movable portion 82 may be movably provided in the internal space 8s of the casing connection portion 81. The casing movable portion 82 may include a first movable piece 83, a second movable piece 84, and a third movable piece 85.

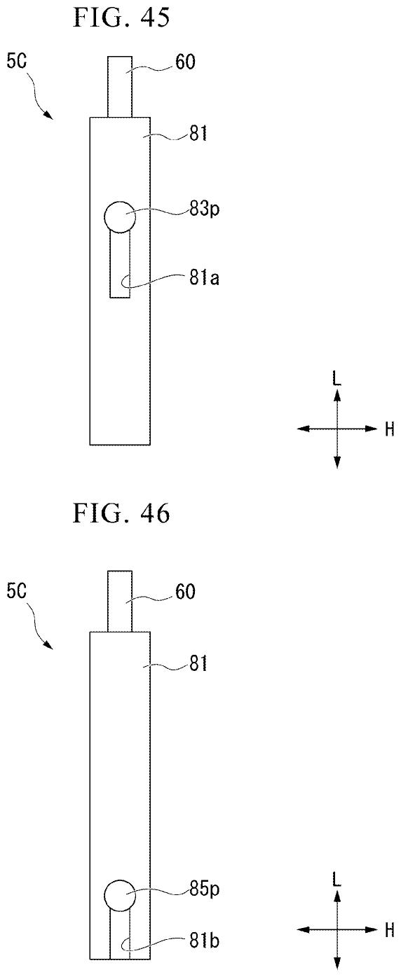

FIG. 45 is a view of the cartridge 5C when viewed from one side in the width direction W.

The first movable piece 83 may include a first path 83a which penetrates the first movable piece 83. The first movable piece 83 may be connected to the second movable piece 84. The first movable piece 83 may include a first stopper 83p which protrudes toward one side in the width direction W. The first stopper 83p can engage with a first slit 81a formed on one side of the casing connection portion 81 in the width direction W and extending in the longitudinal direction L. The first movable piece 83 can move along the first slit 81a in the longitudinal direction L. The first slit 81a may be a slit with both ends in the longitudinal direction L closed and the first stopper 83p does not come off from the first slit 81a.

The second movable piece 84 may include a second path 84a which penetrates the second movable piece 84. The second movable piece 84 may be connected to the first movable piece 83 and the third movable piece 85.

FIG. 46 is a view of the cartridge 5C when viewed from the other side in the width direction W.

The third movable piece 85 may include a third path 85a which penetrates the third movable piece 85. The third movable piece 85 may be connected to the second movable piece 84. The third movable piece 85 may include a third stopper 85p which protrudes toward the other side in the width direction W. The third stopper 85p can engage with a third slit 81b formed on the other side of the casing connection portion 81 in the width direction W and extending in the longitudinal direction L. The third movable piece 85 can move along the third slit 81b in the longitudinal direction L. The proximal end of the first slit 81a in the longitudinal direction L may coincide with the proximal end of the casing connection portion 81 and the first slit can open toward the proximal end side. Thus, the third stopper 85p can be pulled out to the outside of the casing connection portion 81 from the third slit 81b.

The first path 83a, the second path 84a, and the third path 85a can form a path into which the sheath 220 can be inserted (hereinafter, also referred to as the “sheath connection portion 86”).

As shown in FIG. 44, when the first movable piece 83 moves the most distal end side, the first path 83a and the sheath insertion region 64 can communicate with, connect with, contact, or the like each other. When the first movable piece 83 moves toward the most distal end side, the second movable piece 84 and the third movable piece 85 also can move toward the distal end side. At this time, the sheath connection portion 86 (the first path 83a, the second path 84a, and the third path 85a) can form a substantially S-shaped curved path.

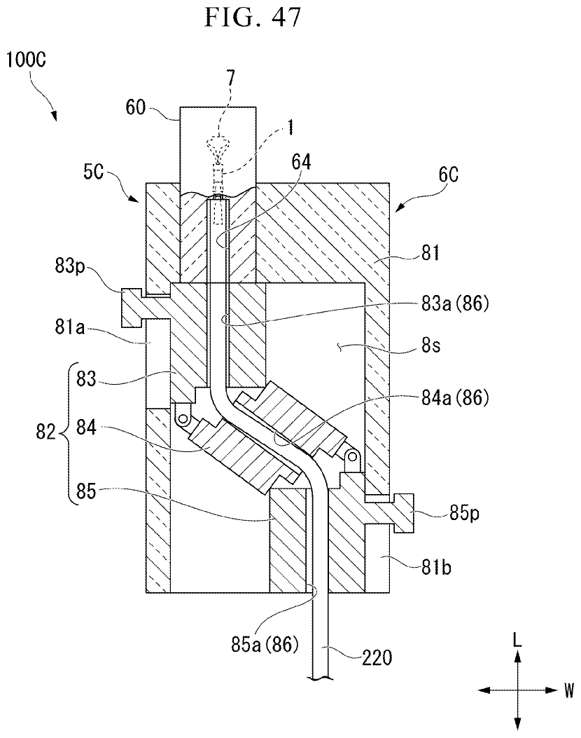

FIG. 47 is a diagram showing the cartridge 5C in which the sheath 220 is inserted into the sheath connection portion 86. When the casing movable portion 82 is disposed on the distal end side of the casing connection portion 81, the sheath connection portion 86 (the first path 83a, the second path 84a, and the third path 85a) can form a substantially S-shaped curved path. That is, when the casing movable portion 82 is disposed on the distal end side of the casing connection portion (or other fixing member or fixing means) 81, the sheath 220 inserted into the cartridge 5C can remain connected to the cartridge 5C not to come off from the cartridge 5C.

The clip unit 1 can be loaded into the clip introducing device 200 by the same method as the first embodiment while the sheath 220 is inserted into the casing main body 60 of the cartridge 5C.

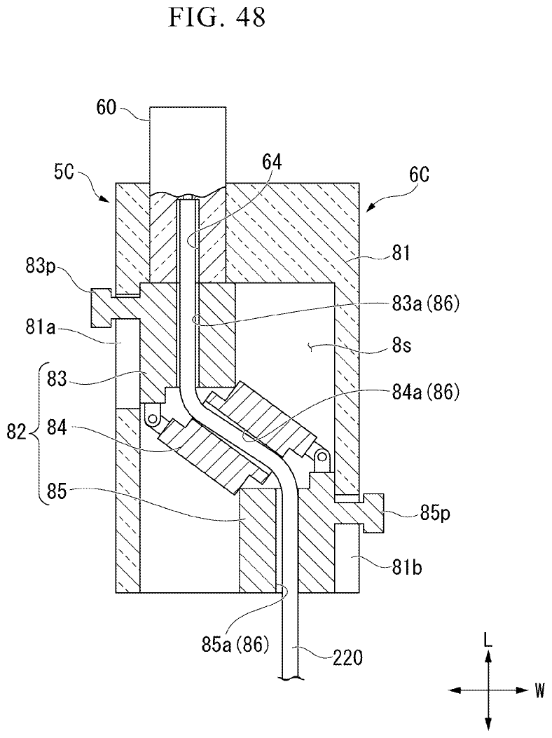

FIGS. 48 to 50 are diagrams showing the casing 6C from which the sheath 220 is pulled out.

When the sheath 220 moves in the direction away from the cartridge 5C, the casing movable portion 82 can move toward the proximal end side together with the sheath 220. The first movable piece 83 can move toward the proximal end side along the first slit 81a. The third movable piece 85 can move toward the proximal end side along the third slit 81b. Until the third stopper 85p is pulled out from the third slit 81b, the sheath connection portion 86 (the first path 83a, the second path 84a, and the third path 85a) can maintain a substantially S-shaped curved path and the sheath 220 may not come off from the cartridge 5C.

Further, when the sheath 220 moves in the direction away from the cartridge 5C, the third movable piece 85 can be separated from the third slit 81b. As a result, as shown in FIG. 50, the sheath connection portion 86 (the first path 83a, the second path 84a, and the third path 85a) may have a linear shape.

The user can straighten the sheath connection portion 86 and pull out the sheath 220 from the casing 6C just by pulling out the sheath 220 from the cartridge 5C.

According to the clip system 500 of this embodiment, the cartridge system 100C and the applicator 200 may be accommodated in the same sterile pack 400 and the cartridge system 100C may be attached to the applicator 200 in advance. Thus, there may be less work to load the clip unit 1 into the applicator 200 using the cartridge system 100C after opening the sterile pack, and the user can use the clip unit 1 loaded into the applicator 200 as soon as possible.

Although the third embodiment of the present disclosure has been described in detail with reference to the drawings, the specific configuration is not limited to this embodiment, and design changes may be made within the scope of the present disclosure. Further, the components shown in the above-described embodiment and the modified examples shown below can be configured by appropriately combining them.

### Modified Example 3-1

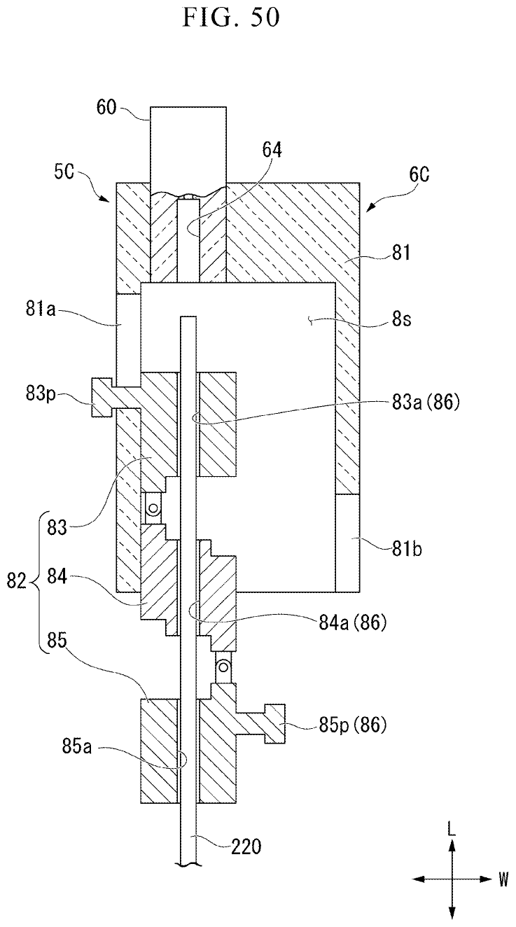

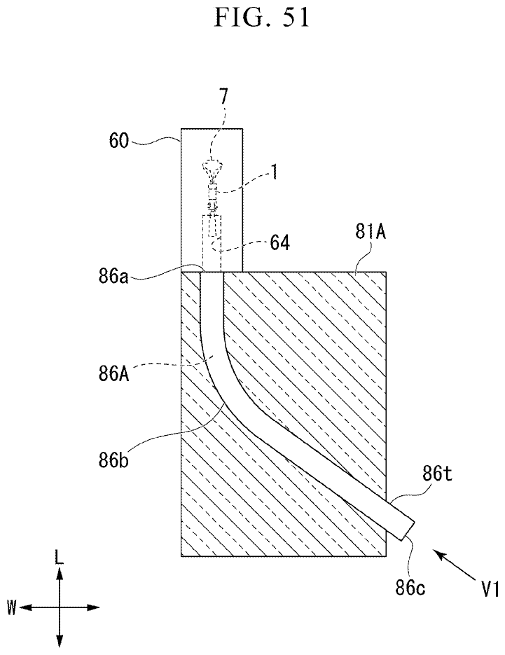

FIG. 51 is a diagram showing a casing connection portion 81A which is a modified example of the casing connection portion 81. The casing connection portion 81A may include a sheath connection portion 86A into which a sheath may be inserted. A tube 86t can be fitted into the sheath connection portion 86A. The sheath connection portion 86A can communicates with, contact, connect with, or the like the sheath insertion region 64 in a distal end opening 86a. The sheath connection portion 86A may include a bent portion 86b bent in the longitudinal direction L (the sheath insertion direction).

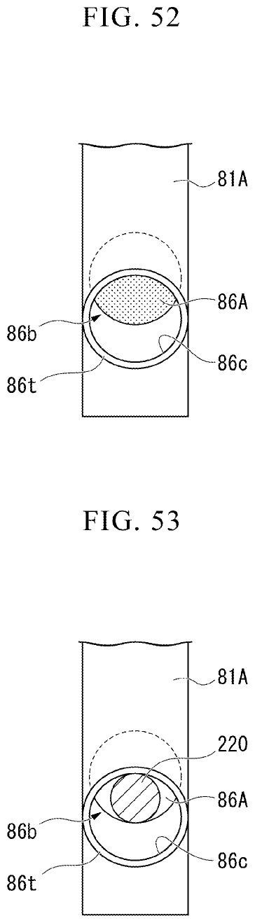

FIGS. 52 and 53 are diagrams showing the casing connection portion 81A when viewed from an arrow V1 of FIG. 51. As shown in FIG. 52, the sheath connection portion 86A in the bent portion 86b may be narrower than the proximal end opening 86c and the like. As shown in FIG. 53, the sheath 220 inserted into the sheath connection portion 86A can remain connected to the cartridge 5C not to come off from the sheath connection portion 86A unless an external force equal to or larger than a predetermined level is applied due to the friction between the surface of the sheath 220 and the surface of the tube 86t.

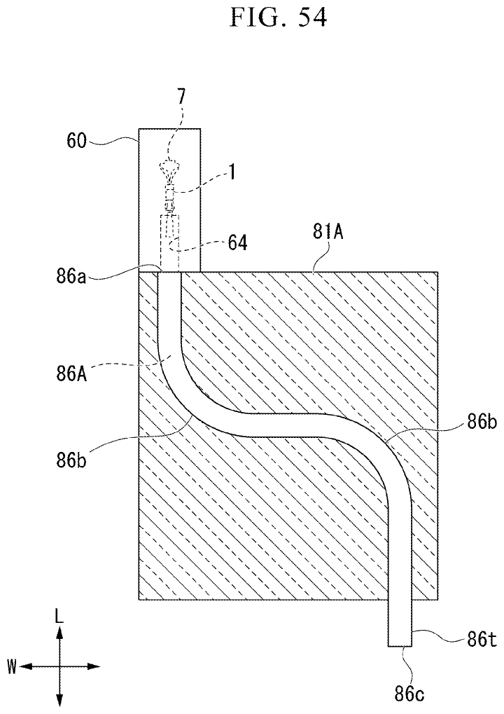

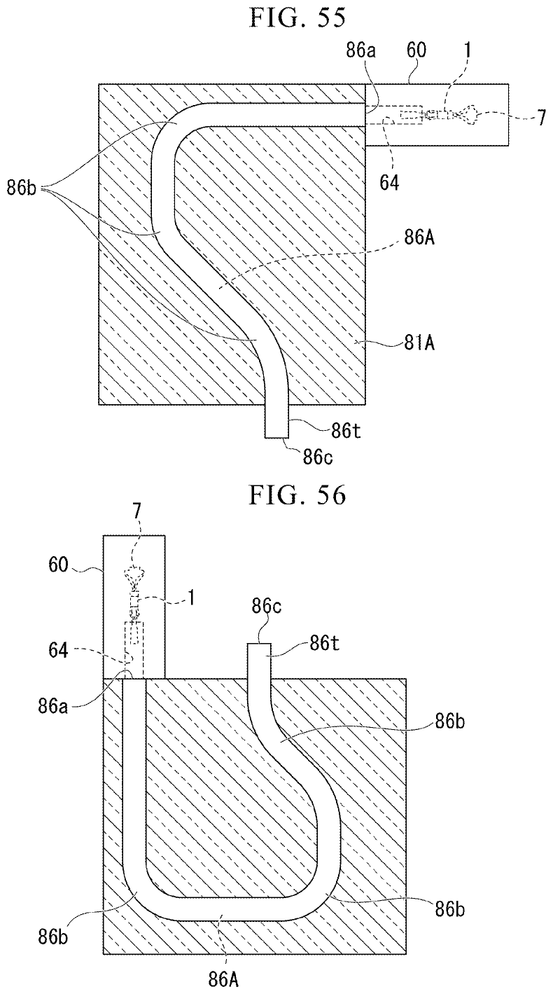

FIGS. 54 to 56 are diagrams showing a modified example of the sheath connection portion 86A. The sheath connection portion 86A may include a plurality of the bent portions 86b. Since two or more bent portions 86b which are bent in different directions are provided, the sheath connection portion 86A can rigidly hold the sheath 220 so that the sheath 220 does not come off. Further, the tube 86t does not need to be fitted into the sheath connection portion 86A as long as the sheath connection portion can sufficiently hold the sheath 220.

### Modified Example 3-2

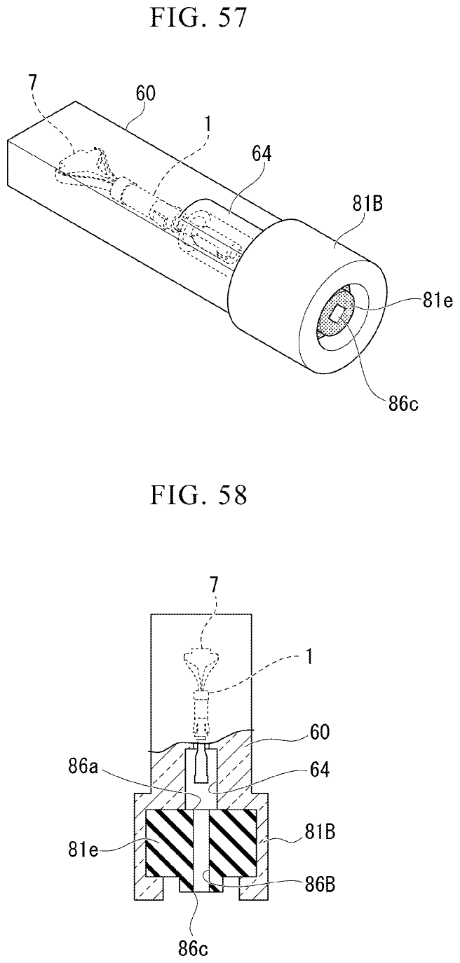

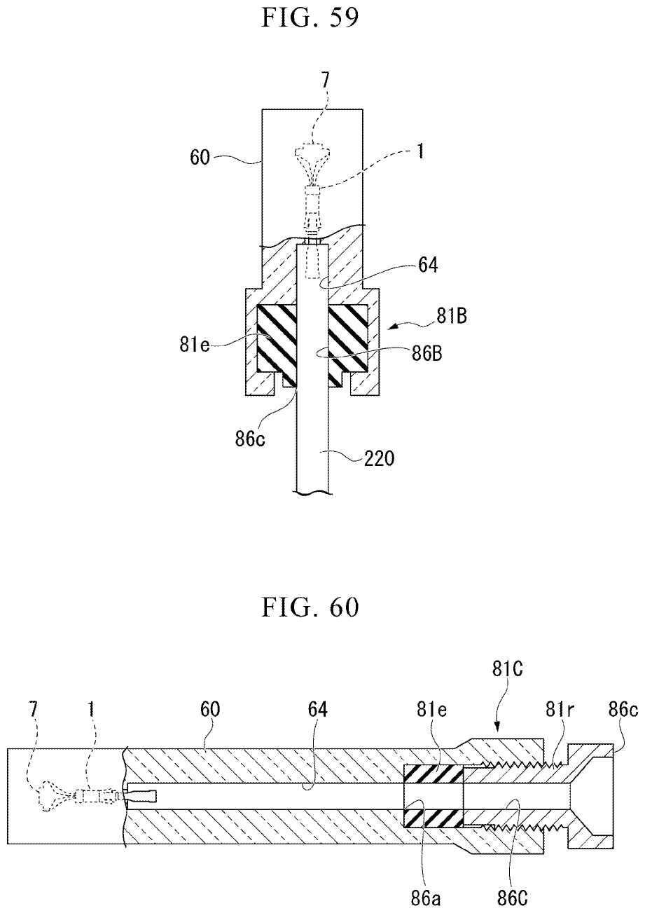

FIGS. 57 to 59 are diagrams showing a casing connection portion 81B which is a modified example of the casing connection portion 81. The casing connection portion 81B may include a cylindrical elastic body 81e and the internal space of the elastic body 81e may be a sheath connection portion 86B. The outer diameter of the sheath 220 inserted from the proximal end opening 86c into the sheath connection portion 86B may be larger than the inner diameter of the sheath connection portion 86B. As shown in FIG. 59, the sheath 220 inserted into the sheath connection portion 86B can remain connected to the cartridge 5C not to come off from the sheath connection portion 86B unless an external force equal to or larger than a predetermined level is applied due to the restoration elastic force of the elastic body 81e of the casing connection portion 81B.

(Modified example 3-3)

FIG. 60 is a diagram showing a casing connection portion 81C which is a modified example of the casing connection portion 81. The casing connection portion 81C may include the cylindrical elastic body 81e and a rotation body 81r which can make the elastic body 81e advance and retract in the longitudinal direction L (the sheath insertion direction). The internal space of the elastic body 81e and the rotation body 81r is the sheath connection portion 86C.

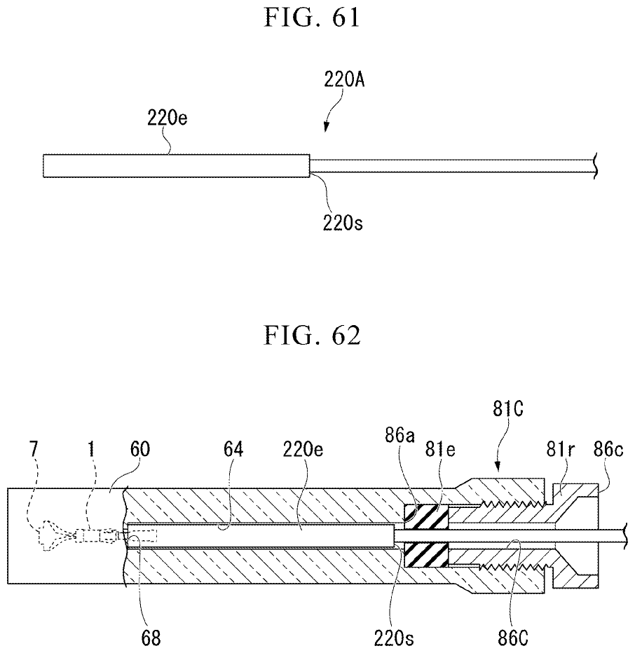

FIG. 61 is a diagram showing a sheath 220A which is a modified example of the sheath 220. The sheath 220A includes an enlarged diameter portion 220e at the distal end so that the outer diameter on the proximal end side may be larger than that on the distal end side. A step 220s may be formed at the proximal end of the enlarged diameter portion 220e.

FIG. 62 is a diagram showing the casing connection portion 81C into which the sheath 220A is inserted. When the sheath 220A contacts the sheath contact portion 68, the step 220s may be located on the distal end side rather than the elastic body 81e. When the rotation body 81r is rotated to advance as shown in FIG. 62, the elastic body 81e may be compressed in the longitudinal direction L and the inner diameter of the internal space can decrease to be in a close contact state with the sheath 220A. Since the step 220s is caught by the elastic body 81e, the sheath 220 inserted into the sheath connection portion 86C can remain connected to the cartridge 5C not to come off from the sheath connection portion 86C unless an external force equal to or larger than a predetermined level is applied.

### Modified Example 3-4

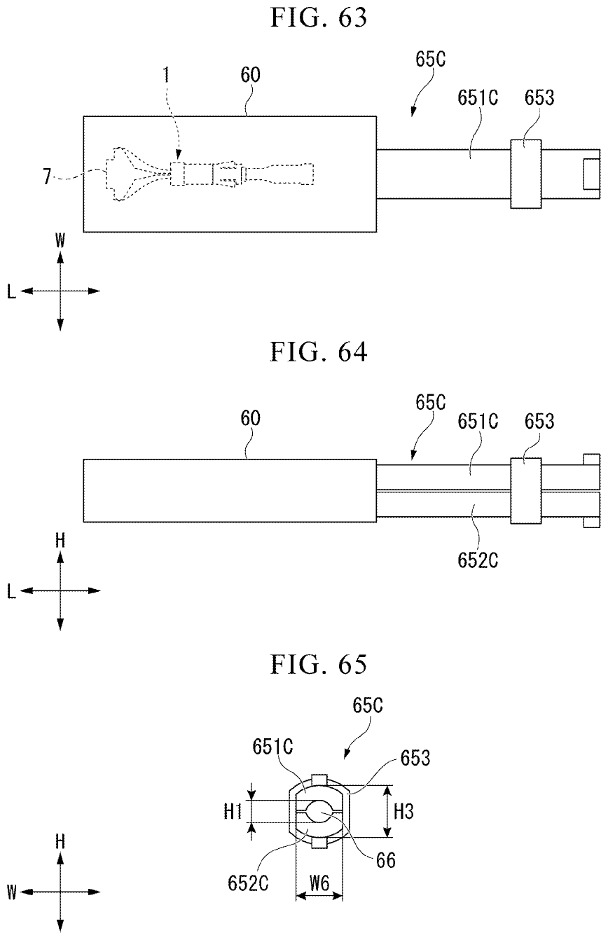

FIGS. 63 to 67 are diagrams showing a compressing portion 65C which is a modified example of the compressing portion 65B.

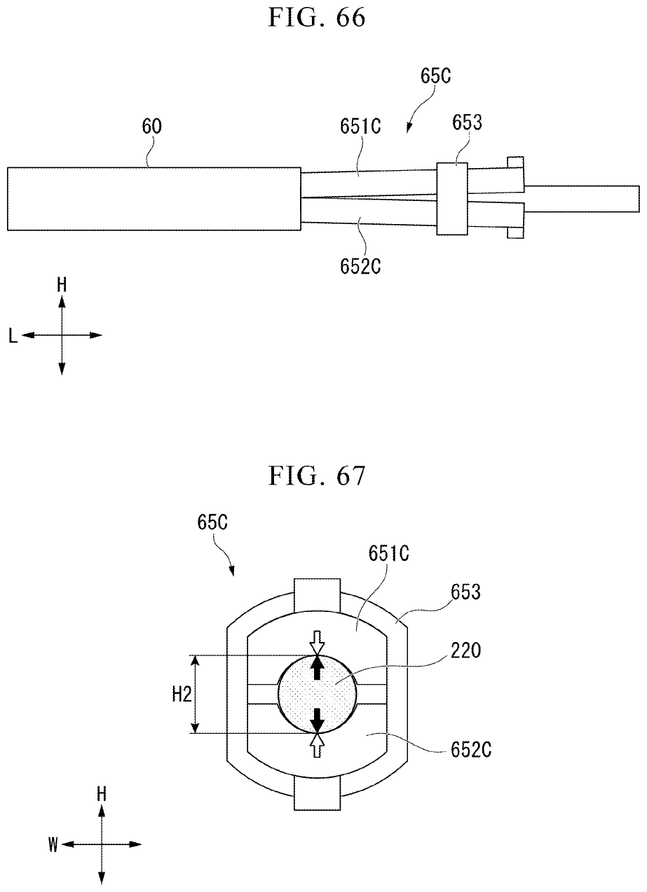

The compressing portion 65C may be a plate-shaped member provided at the proximal end of the casing main body 60. The compressing portion 65C may include a first compressing portion 651C, a second compressing portion 652C, and an elastic ring 653. The first compressing portion 651C and the second compressing portion 652C may be provided or arranged to face each other in the height direction H with the sheath connection portion 66 interposed or located therebetween. The elastic ring 653 is provided to bring the first compressing portion 651C and the second compressing portion 652C closer together. Thus, the height H1 of the internal space of the sheath connection portion 66 may be smaller than the outer diameter H2 of the sheath 220. Thus, as shown in FIGS. 66 and 67, when the sheath 220 is inserted into the sheath connection portion 66, the sheath 220 can be sandwiched or located between the first compressing portion 651C and the second compressing portion 652C not to come off from the sheath connection portion 66. That is, the sheath 220 inserted into the sheath connection portion 66 of the compressing portion 65C remains connected to the cartridge 5C not to come off, separate, or disconnect from the sheath connection portion 66 of the compressing portion 65C unless an external force equal to or larger than a predetermined level is applied. Further, the height H3 in the height direction H (the opening/closing direction) of the compressing portion 65C contacting the elastic ring 653 may be larger than the length W6 of the width direction W of the compressing portion 65C contacting the elastic ring 653.

### Fourth Embodiment

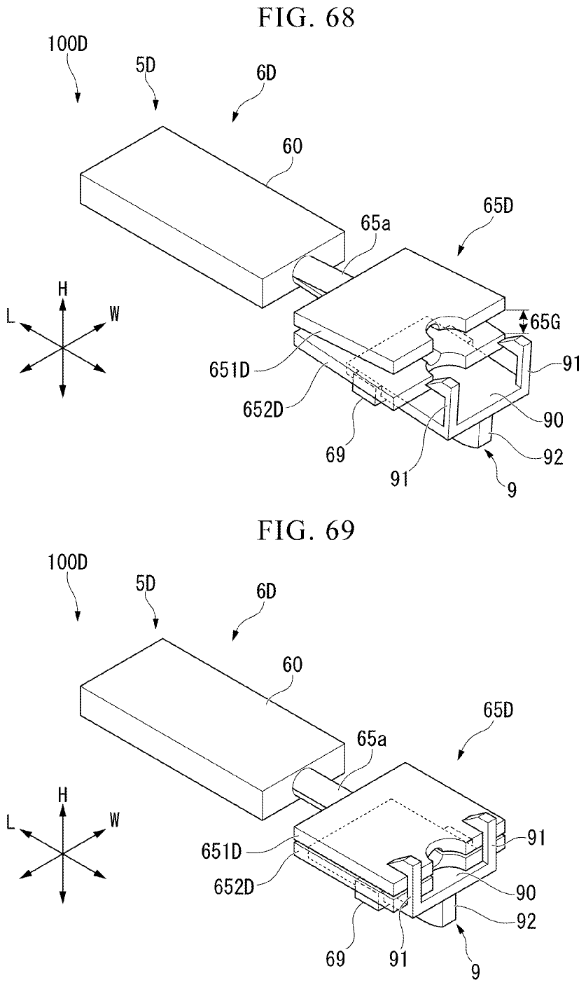

A fourth embodiment of the present disclosure will be described with reference to FIGS. 68 to 78. In the following description, components that are common to those already described will be given the same reference numerals and redundant description will be omitted.

FIG. 68 is a diagram showing a cartridge system 100D according to a fourth embodiment. Similar to the cartridge system 100 of the first embodiment, the cartridge system 100D may be packaged in the sterile pack 400 as the clip system 500 together with the clip introducing device (applicator) 200. The cartridge system 100D may include the clip unit 1 and a cartridge 5D which can accommodate or store the clip unit 1.

The cartridge 5D may include a casing 6D, the regulation member 7, and a locking mechanism 9. The cartridge 5D can accommodate or store the clip unit 1.

Similar to the casing 6 of the first embodiment, the casing 6D can store the clip unit 1 together with the regulation member 7. The casing 6D may include the casing main body 60, a compressing portion 65D, the sheath connection portion 66, and a locking mechanism support portion 69.

The compressing portion 65D may be a plate-shaped member provided at the proximal end of the casing main body 60. The compressing portion 65D may include a first compressing portion 651D and a second compressing portion 652D. The first compressing portion 651D and the second compressing portion 652D may be provided to face each other in the height direction H with the sheath connection portion 66 interposed or located therebetween.

The compressing portion 65D may include the connection portion 65a connected to the casing main body 60. The connection portion 65a can individually connect the first compressing portion 651D and the second compressing portion 652D to the casing main body 60. The connection portion 65a can be bent so that the first compressing portion 651D and the second compressing portion 652D are separated from each other. Thus, the gap 65G can be formed between the first compressing portion 651D and the second compressing portion 652D. The first compressing portion 651D and the second compressing portion 652D can be further separated from each other on the proximal end side rather than the distal end side.

The locking mechanism support portion 69 may be provided on one side (e.g., a lower side) in the height direction H of the second compressing portion 652D. The locking mechanism support portion 69 can support the locking mechanism 9 attached to the compressing portion 65D.

The locking mechanism (or other fixing member or fixing means) 9 may include a main body portion 90, a claw 91, a knob (first knob) 92. The main body portion 90 may be formed in a substantially plate shape. The main body portion 90 may be supported by the locking mechanism support portion 69. The claw 91 may be provided at the proximal end of the main body portion 90 and protrude toward the other side (e.g., an upper side) in the height direction H. Two claws 91 may be provided on both sides in the width direction W. The knob 92 may be a convex portion which protrudes from the main body portion 90 toward one side (e.g., the lower side) in the height direction H.

FIG. 69 is a diagram showing the cartridge 5D in which the locking mechanism 9 is attached to the compressing portion 65D. When the locking mechanism 9 is attached to the compressing portion 65D, the claw 91 can sandwich the first compressing portion 651D and the second compressing portion 652D and holds the first compressing portion 651D and the second compressing portion 652D at a predetermined interval.

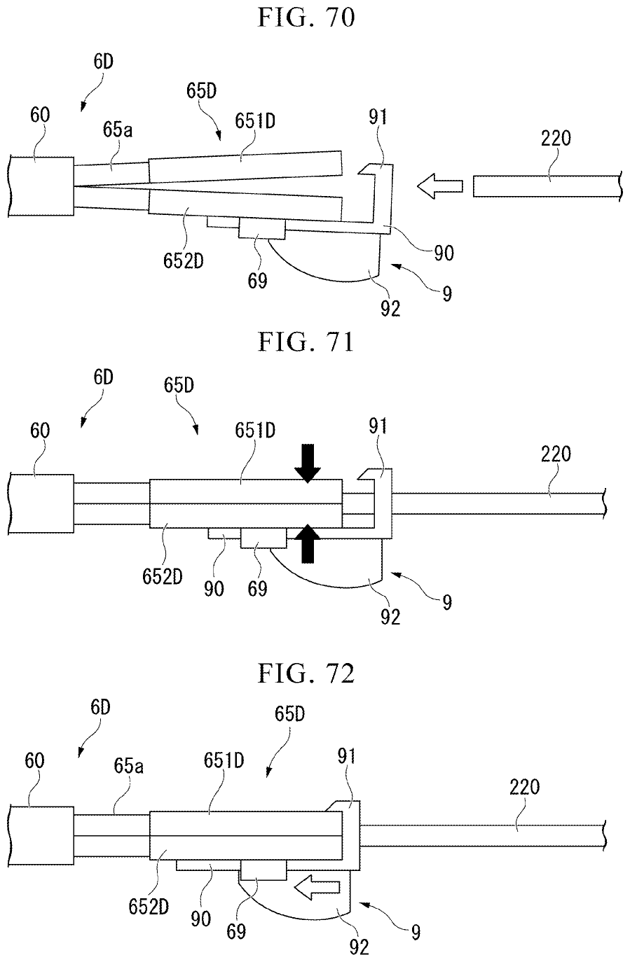

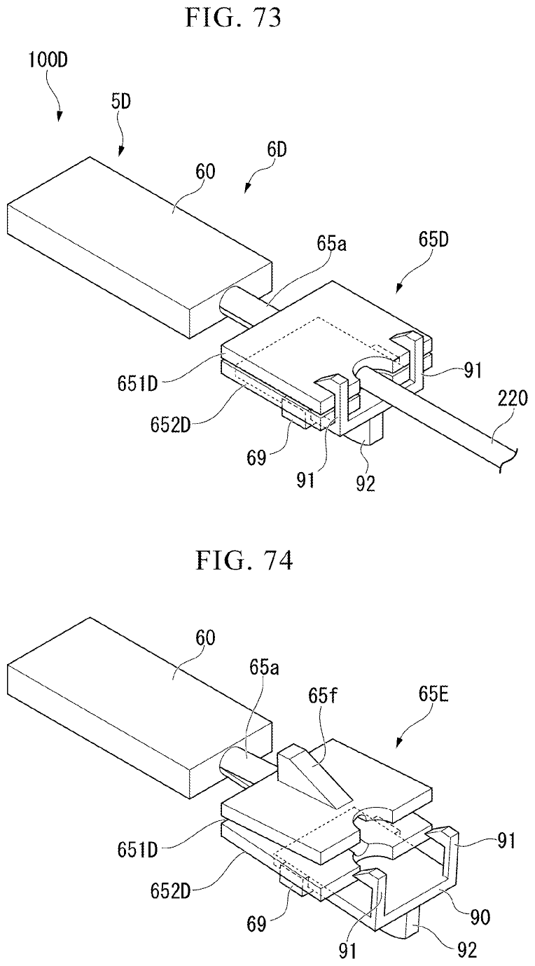

FIGS. 70 to 73 are diagrams illustrating a method of fixing the sheath 220 so that the sheath does not come off from the cartridge 5D. As shown in FIG. 70, the user can insert the sheath 220 into the sheath connection portion 66 of the compressing portion 65D to which the locking mechanism 9 is not attached. Next, as shown in FIG. 71, the user can sandwich the first compressing portion 651D and the second compressing portion 652D and bring them closer together. Next, the user can slide the locking mechanism 9 toward the distal end side as shown in FIG. 72 and hook the locking mechanism onto the first compressing portion 651D and the second compressing portion 652D with the claw 91 as shown in FIG. 73. When the locking mechanism (or other fixing member or fixing means) 9 is attached to the compressing portion 65D, the sheath 220 inserted into the cartridge 5D can remain connected to the cartridge 5D so as not to come off or separate from the cartridge 5D.

The clip unit 1 may be loaded into the clip introducing device 200 by the same method as the first embodiment while the sheath 220 is inserted into the casing main body 60 of the cartridge 5D.

FIG. 74 is a diagram showing a compressing portion 65E which is a modified example of the compressing portion 65D.

The compressing portion 65E may include a knob (second knob) 65f which protrudes from the first compressing portion 651D toward the other side (upper side) in the height direction H. The knob 92 of the locking mechanism 9 may include a slope directed toward the distal end side in the longitudinal direction L. On the other hand, the knob 65f of the compressing portion 65E may include a slope directed toward the proximal end side in the longitudinal direction L.

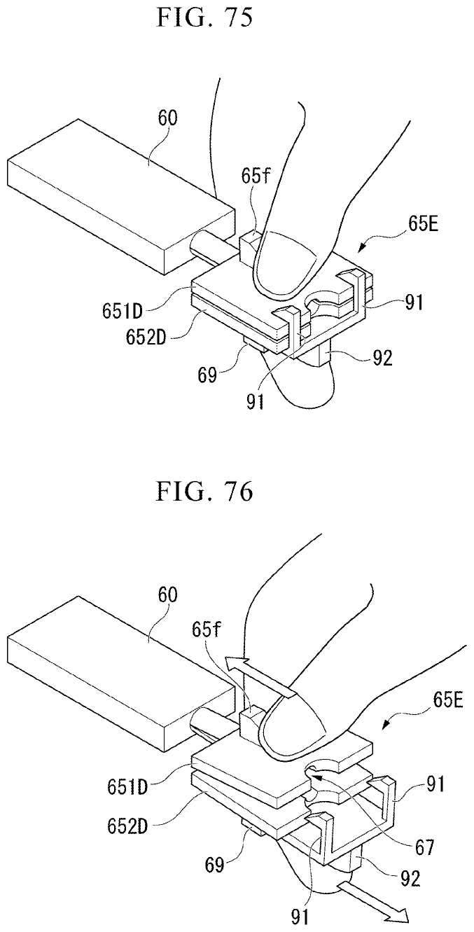

FIGS. 75 and 76 are diagrams illustrating a method of separating the locking mechanism 9 from the casing 6D. As shown in FIG. 75, the user can separate the locking mechanism 9 from the casing 6D by grasping the knob 65f and the knob 92 with their fingers and sliding them in the direction indicated by the arrow shown in FIG. 76. When the locking mechanism 9 is separated from the casing 6D, the sheath insertion port 67 can be expanded and the sheath 220 can be inserted into and removed from the sheath connection portion 66.

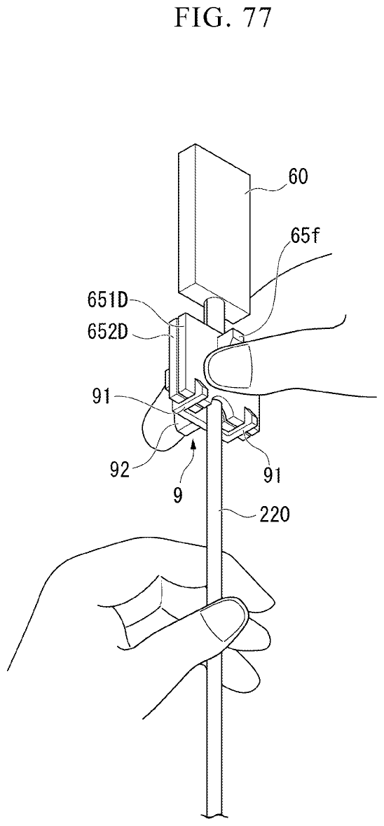

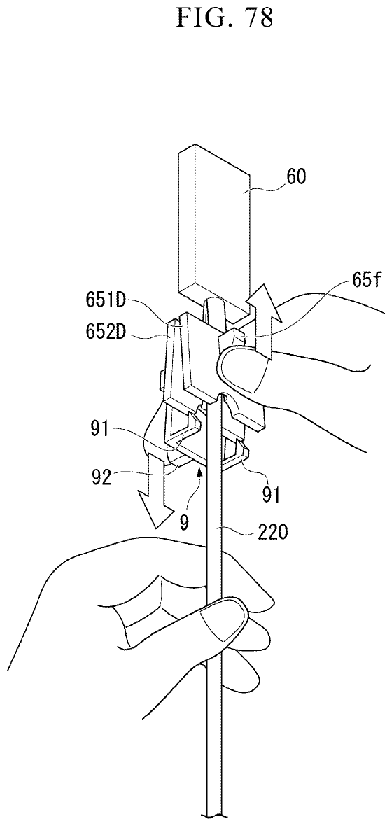

FIGS. 77 and 78 are diagrams illustrating a method of removing the cartridge 5D from the sheath 220. If the knob 65f and the knob 92 are respectively held by an index finger and a thumb as shown in FIG. 77 and are moved in the direction indicated by the arrow shown in FIG. 78 when holding the cartridge 5D after loading the clip unit 1 into the applicator 200, the cartridge 5D can be held and the cartridge 5D can be removed from the sheath 220.

According to the clip system 500 of this embodiment, the cartridge system 100D and the applicator 200 can be accommodated or stored in the same sterile pack 400 and the cartridge system 100D can be attached to the applicator 200 in advance. Thus, there may be less work to load the clip unit 1 into the applicator 200 using the cartridge system 100D after opening the sterile pack, and the user can use the clip unit 1 loaded into the applicator 200 as soon as possible.

Although the fourth embodiment of the present disclosure has been described in detail with reference to the drawings, the specific configuration is not limited to this embodiment, and design changes may be made within the scope of present disclosure. Further, the components shown in the above-described embodiment and the modified examples shown below can be configured by appropriately combining them.

### Modified Example 4-1

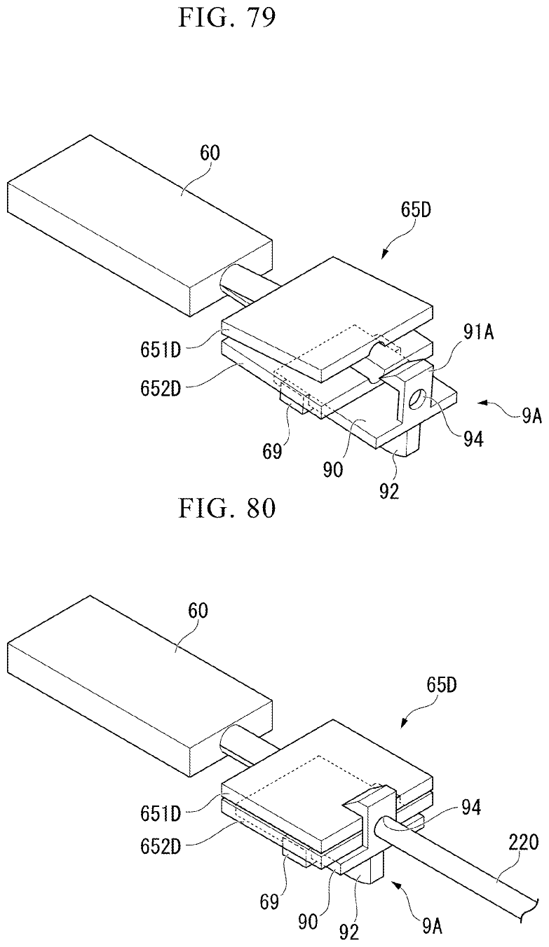

FIGS. 79 and 80 are diagrams showing a locking mechanism 9A which is a modified example of the locking mechanism 9. The locking mechanism 9A may include the main body portion 90, a claw 91A, and the knob 92. The claw 91A may be provided at the proximal end of the main body portion 90 and protrude toward the other side (upper side) in the height direction H. One claw 91 may be provided at the center in the width direction W. The claw 91A may be provided with an insertion or extraction hole 94 into which the sheath 220 can be inserted.

### Modified Example 4-2

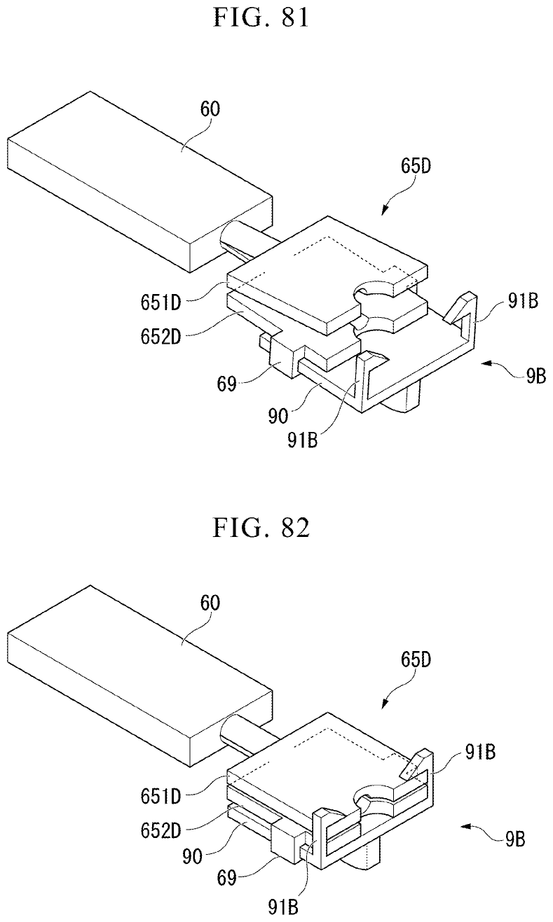

FIGS. 81 and 82 are diagrams showing a locking mechanism 9B which is a modified example of the locking mechanism 9. The locking mechanism 9B may include the main body portion 90, a claw 91B, and the knob 92. Two claws 91B may be provided on both sides in the width direction W. Two claws 91B may be located at positions that sandwich the first compressing portion 651D and the second compressing portion 652D from the outside in the width direction W.

While embodiments of the disclosure have been described and illustrated above, it should be understood that these are exemplary of the disclosure and are not to be considered as limiting. Additions, omissions, substitutions, and other modifications can be made without departing from the scope of the present disclosure. Accordingly, the disclosure is not to be considered as being limited by the foregoing description, and is only limited by the scope of the claims.

The present disclosure can be applied to a cartridge and the like storing a clip unit.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A clip system comprising:
a clip;
a cartridge configured to store the clip;
an applicator into which the clip is configured to be loaded; and
a pack packing the clip, the cartridge and the applicator together.

2. The clip system according to claim 1, wherein the cartridge and the applicator are packaged in the pack in a connected state.

3. The clip system according to claim 1, wherein the cartridge and the applicator are packaged in the pack in a separated state.

4. The clip system according to claim 1, wherein the cartridge includes:
a fixing body connecting with the applicator so that the clip is configured to be loaded into the applicator.

5. The clip system according to claim 4, wherein the cartridge further includes:
a hole configured to receive a sheath of the applicator, and wherein the fixing body includes a compressing portion configured to sandwich the hole.

6. The clip system according to claim 4, wherein the cartridge further includes:
a hole configured to receive a sheath of the applicator; and
a compressing portion configured to sandwich the hole, wherein the fixing body includes an outer casing configured to contain at least the compressing portion.

7. The clip system according to claim 4, wherein the cartridge further includes:
a hole configured to receive a sheath of the applicator, and wherein the fixing body includes a bent portion provided in the sheath connection portion.

8. The clip system according to claim 4, wherein the cartridge further includes:
a hole configured to receive a sheath of the applicator; and
a compressing portion which sandwiches the sheath connection portion, and wherein the fixing body includes a locking mechanism configured to sandwich the compressing portion.

9. A clip system comprising:
a clip;
a cartridge configured to store the clip; and
an applicator into which the clip is configured to be loaded, wherein the cartridge includes a fixing body connecting with the applicator so that the clip is configured to be loaded into the applicator.

10. The clip system according to claim 9, wherein the cartridge includes:
a hole configured to receive a sheath of the applicator, and wherein the fixing body includes a compressing portion configured to sandwich the sheath connection portion.

11. The clip system according to claim 9, wherein the cartridge includes:
a hole configured to receive a sheath of the applicator; and
a compressing portion configured to sandwich the sheath connection portion, wherein the fixing body includes an outer casing configured to contain at least the compressing portion.

12. The clip system according to claim 9, wherein the cartridge includes:
a hole configured to receive a sheath of the applicator, and wherein the fixing body includes a bent portion provided in the sheath connection portion.

13. The clip system according to claim 9, wherein the cartridge system includes:
a hole configured to receive a sheath of the applicator; and
a compressing portion configured to sandwich the sheath connection portion, wherein the fixing body includes a locking mechanism configured to sandwich and fix the compressing portion.

14. A method of loading a clip, comprising:
opening a cartridge and an applicator from a pack, while a connection between the cartridge and the applicator is maintained;
loading the clip into the applicator; and
releasing the connection between the cartridge and the applicator.

15. The method according to claim 14, wherein
the releasing the connection between the cartridge and the applicator comprises manually expanding a compressing portion sandwiching a sheath connection portion into which a sheath of the applicator is inserted.

16. The method according to claim 15, wherein
the releasing the connection between the cartridge and the applicator comprises pulling out the sheath from a sheath connection portion into which a sheath of the applicator is inserted.

17. The method according to claim 14, wherein
the releasing the connection between the cartridge and the applicator comprises removing a locking mechanism configured to sandwich and fix a compressing portion.

18. The method according to claim 17, wherein the compressing portion is configured to sandwich a sheath connection portion into which a sheath of the applicator is inserted.

19. The clip system according to claim 1, wherein:
the applicator includes:
a sheath;
a wire inserted into the sheath; and
a connector located at a distal end of the wire, the connector configured to connect the applicator and the clip,
the cartridge includes;
a storing body having a container configured to store the clip; and
a compressing portion configured to fix the sheath and the storing body, the compressing portion including a first compressing portion and a second compressing portion, the first compressing portion having an open state and a closed state relative to the second compressing portion, the first compressing portion and the second compressing portion biased to the closed state, the first compressing portion and the second compressing portion sandwiching the sheath.

20. The clip system according to claim 1, wherein:
the applicator includes:
a sheath;
a wire inserted into the sheath; and
a connector located at a distal end of the wire, the connector configured to connect the applicator and the clip,
the cartridge includes;
a storing body having a container configured to store the clip;
a compressing portion configured to fix the sheath and the storing body, the compressing portion including a first compressing portion and a second compressing portion, the first compressing portion having an open state and a closed state relative to the second compressing portion, the first compressing portion and the second compressing portion sandwiching the sheath; and
an outer case configured to overlap the storing body and the compressing portion, the outer case fixing the first compressing portion and the second compressing portion in the closed state.
